SEQUENCE LISTING (4 sequences)

SEQ ID NO: 1 - DNA, 7013 bp, Artificial Sequence
  Recombinant vector or recombinant plasmid
  misc_feature (18)..(791): Linked\1D3\variable\regions
  misc_feature (793)..(1103): CD28
  misc_feature (1104)..(1445): TCR-zeta
  LTR (1516)..(2030): 3′\LTR
  misc_feature (3339)..(4200): Coding Region
  LTR (5203)..(5716): 5′\LTR
  misc_signal (5779)..(5792): Splicing donor
  misc_signal (6728)..(6752): Splicing Accetor
tcgagaagcttgccaccatgggtgtccctacccagctcctgggactgctcctgctgtggatcaccgacgccatctgcgacatccagatgacccagagccctgccagcctgtctaccagcctgggcgagacagtgaccatccagtgtcaggccagcgaggacatctactctggcctggcttggtatcagcagaagcccggcaagagccctcagctgctgatctacggcgccagcgacctgcaggacggcgtgcctagcagattcagcggcagcggctccggaacccagtacagcctgaagatcaccagcatgcagaccgaggacgagggcgtgtacttctgccagcaaggcctgacctaccctagaaccttcggaggaggcaccaagctggaactgaagggcggaggcggaagtggaggcggaggatctggcggcggaggctctgaagtgcagctgcagcagtctggcgctgaactggtccggcctggcactagcgtgaagctgtcctgcaaggtgtccggcgacaccatcaccttctactacatgcacttcgtgaagcagaggccaggacagggcctggaatggatcggcagaatcgaccctgaggacgagagcaccaagtacagcgagaagttcaagaacaaggccaccctgaccgccgacaccagcagcaacaccgcctacctgaagctgtctagcctgacctccgaggacaccgccacctacttttgcatctacggcggctactacttcgactactggggccagggcgtgatggtcaccgtgtccagcatcgagttcatgtacccccctccctacctggacaacgagagaagcaacggcaccatcatccacatcaaagaaaagcacctgtgccacacccagagcagccccaagctgttctgggccctggtggtggtggccggcgtgctgttctgttacggcctgctggtcacagtggccctgtgcgtgatctggaccaacagcagaagaaacagaggcggccagagcgactacatgaacatgacccccagaaggccaggcctgaccagaaagccctaccagccctacgcccctgccagagacttcgccgcctacagacccagagccaagttcagcagatccgccgagacagccgccaacctgcaggatcccaaccagctgttcaacgagctgaacctgggcagacgggaggaattcgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgttccagggcctgtccaccgccaccaaggacaccttcgacgccctgcacatgcagaccctggcccccagatgagtcgacggtaccgcgggcccgggatccgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatccatcgatggagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgatttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcctagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgc

SEQ ID NO: 2 - DNA, 18 bp, Mus musculus
gacttcgccgcctacaga

SEQ ID NO: 3 - DNA, 19 bp, Mus musculus
tcgttgaacagctggttgg

SEQ ID NO: 4 - protein, 258 aa, Rattus rattus
MGVPTQLLGLLLLWITDAICDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKGGGGSGGGGSGGGGSEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSS

(19) United States
(12) Patent Application Publication
(10) Pub. No.: US 2020/0085871 A1
(43) Pub. Date: Mar. 19, 2020

(54) METHODS OF USING CYTOTOXIC T CELLS FOR TREATMENT OF AUTOIMMUNE DISEASES

(71) Applicant: University of Tennessee Research Foundation, Knoxville, TN (US)

(72) Inventor: Marko Z. RADIC, Germantown, TN (US)

(21) Appl. No.: 16/494,484

(22) PCT Filed: Mar. 19, 2018
(86) PCT No.: PCT/US18/23125

§ 371 Date: Sep. 16, 2019

Related U.S. Application Data
(60) Provisional application No. 62/472,709, filed on Mar. 17, 2017.

(51) Int. Cl. A61K 35/17; C07K 16/28; A61P 37/00

(52) U.S. Cl. CPC A61K 35/17; A61P 37/00; C07K 16/2803

(57) ABSTRACT

The present disclosure relates to methods of using engineered cytotoxic T cells comprising a recombinant vector construct that expresses a chimeric antigen receptor to reduce and/or deplete the number of B cells in a subject in order to treat autoimmune diseases, such as lupus.

### CROSS-REFERENCE TO RELATED APPLICATIONS

This application claims priority under 35 U.S.C. § 119 of U.S. Provisional Patent Application Ser. No. 62/472,209 filed Mar. 17, 2017, the entirety of which is incorporated herein by reference.

### REFERENCE TO SEQUENCE LISTING SUBMITTED ELECTRONICALLY

The official copy of the sequence listing is submitted electronically via EFS-Web as an ASCII formatted sequence listing with a file named “274929_ST25.txt,” created on Mar. 19, 2018, having a size of 12.4 kilobytes, and is filed concurrently with the specification. The sequence listing contained in this ASCII formatted document is part of the specification and is herein incorporated by reference in its entirety.

### FIELD OF THE PRESENT APPLICATION

The present disclosure relates to methods of using engineered cytotoxic T cells comprising a recombinant vector construct that expresses a chimeric antigen receptor to reduce and/or deplete the number of B cells in a subject in order to treat autoimmune diseases, such as lupus.

### BACKGROUND

Lupus and rheumatoid arthritis are two of the most common and severe autoimmune diseases, affecting over 1.5 million American people annually. Both conditions result due to the malfunction of the immune system, which mistakenly attacks parts of the body that it is designed to protect. While treatments of autoimmune diseases have historically been aimed at decreasing inflammation and pain that often results, there still remains no cure for most autoimmune diseases. Thus, improved treatments for autoimmune disorders are still being heavily pursued in both commercial and university research and development organizations.

It is known that B cells are primarily responsible for the cellular response observed in autoimmune diseases, such as lupus. More specifically, B cells produce the autoantibodies that are inappropriately directed against the body of a subject or a patient and that mediate immune complex deposition in tissues. Notably, B cells are activated by specific nuclear autoantigen complexes that stimulate both the antigen receptor and the toll-like receptors in B cells.

In addition, B cells act as efficient antigen-presenting cells that, if activated, can stimulate naive T cells to respond to antigen, thus amplifying the anti-self-response. Therefore, B cells carry out essential functions that induce and promote the autoimmune response in lupus. For this reason, B cells are a common target of immunotherapies that are currently approved or in development for autoimmune diseases, including lupus.

B cell depletion therapy in leukemia and lymphoma patients has been hailed as a major achievement in cancer immunotherapy, and results from a growing number of cancer patients indicate that total B cell eradication in patients is possible and can lead to lasting disease remission. This premise applied to cancer therapy development was recently applied in a new human clinical treatment of lupus. Namely, Rituximab, a monoclonal antibody (MAb) to the B cell surface molecule, CD20, was recently developed as a lupus therapy. This MAb treatment, originally used to treat B cell cancer malignancies, effectively lowers the number of circulating B cells in the subject. In lupus trials, Rituximab was highly effective in certain systemic lupus erythematosus (SLE) patients as it reduced B cell numbers and, in consequence, the levels of serum autoantibodies.

Large-scale clinical trials tested the effectiveness of the Rituximab treatment for lupus in humans, however, the results were disappointing. Rituximab failed to achieve the desired efficacy in clinical trials. The negative results prompted different interpretations, including the conclusion that inadequate response indicated poor trial design or patient selection. In some instances, patients responded favorably to anti-CD20 antibody, suggesting that patient-specific conditions may determine efficacy of the CD20 treatment.

One possibility leading to the lackluster results of the Rituximab human clinical trials was that control patients who did not receive Rituximab were treated with high-dose corticosteroids. The steroid treatment may have blurred the beneficial effect of Rituximab treatment because it is known to significantly improved lupus symptoms. In fact, since that time, smaller numbers of lupus patients have continued to receive Rituximab. Results indicate that B cell depletion can be beneficial for lupus, as well as other autoimmune conditions.

An alternative explanation for the reduced effectiveness of the anti-CD20 treatment of Rituximab was uncovered by using autoimmune mice expressing the human CD20 marker. Careful analysis revealed that the anti-CD20 treatment was less effective at reducing B cell numbers in autoimmune mice than in non-autoimmune mice. This reduced sensitivity to anti-CD20 treatment could be attributed to reduced IgG-dependent phagocytosis of the opsonized B cells.

An additional possibility is that in autoimmune mice, and by extrapolation in human lupus patients, anti-CD20 is less effective because of the high levels of endogenous IgG and a subsequent decrease in phagocytosis by macrophage and neutrophils. An additional drawback of Rituxumab is that, being a monoclonal antibody, it decays over time and requires repeated injections of the recombinant protein. This is because an injected monoclonal antibody follows drug-like pharmacokinetics with exponential decay that necessitates repeated administrations to achieve a therapeutic dose. Over time, resistance to the treatment arises in the host and further limits use.

Even though B cell depletion is remarkably long lived, previous results indicate that CD20-treated B cells recover to normal or previous levels and additional and/or subsequent CD20 treatments become necessary. Thus, to date B cell depletion therapy has not been shown to maintain stable and/or long-term reduction of B cell populations, particularly without subsequent and/or additional administrations of treatment.

Moreover, because the anti-CD20 protein of the Rituxumab therapy is derived from a mouse, it elicits an immune response to the foreign protein in humans. This observation may explain why some human patients become refractory to Rituxumab therapy with time, and thus lose efficacy. Accordingly, while Rituxumab has shown an advance in lupus treatment, the industry has not yet reached its potential. The present disclosure is directed to methods of treating autoimmune diseases, such as lupus, and clinical manifestations of such autoimmune diseases using engineered cytotoxic T cells.

### SUMMARY OF THE INVENTION

The present disclosure is directed to a method of treating an autoimmune disease in a subject. The method of treating an autoimmune disease in a subject comprises administering a plurality of engineered cytotoxic T cells to the subject, wherein each of the plurality of engineered cytotoxic T cells comprise a recombinant vector that expresses a chimeric antigen receptor. The method further comprises depleting the number of antibody-producing cells in the subject. In addition, the method comprises improving one or more clinical manifestations of the autoimmune disease in the subject, and treating the autoimmune disease in the subject.

The autoimmune disease of the present method may be Systemic Lupus Erythematosus (SLE). The subject of the present method may be a mammal, wherein the mammal is a mouse or a human. The antibody-producing cells of the present method may be B cells. One or more clinical manifestations of the autoimmune disease in the subject of the present method comprise an increase in B cells. Finally, the chimeric antigen receptor (CAR) of the present method comprises anti-CD19.

Another embodiment of the present method is directed to treating one or more clinical manifestations of lupus in a subject. The method comprises administering a plurality of engineered cytotoxic T cells to the subject, wherein each of the plurality of engineered cytotoxic T cells comprise a recombinant vector that expresses a chimeric antigen receptor. The method further comprises reducing or depleting the number of antibody-producing cells in the subject. The method also comprises preventing, delaying, or reversing one or more clinical manifestations of lupus in the subject. Thus, this embodiment of the claimed method is directed to treating the one or more clinical manifestations of lupus in the subject.

The autoimmune disease of the present method may be Systemic Lupus Erythematosus (SLE). The subject of the present method may be a mammal, wherein the mammal is a mouse or a human. The antibody-producing cells of the present method may be B cells. One or more clinical manifestations of the autoimmune disease in the subject of the present method comprise an increase in B cells. Finally, the chimeric antigen receptor (CAR) of the present method comprises anti-CD19.

Yet another embodiment of the present method comprises monitoring efficacy of lupus treatment in a subject. The method also comprises measuring one or more clinical manifestations of lupus in the cells and/or tissues of a subject prior to treatment administration. The method further comprises administering a treatment construct comprising a plurality of engineered cytotoxic T cells to the subject, wherein each of the plurality of engineered cytotoxic T cells comprise a recombinant vector that expresses a chimeric antigen receptor. At least one hour after treatment administration, this method comprises remeasuring the one or more clinical manifestations of lupus in the subject. Additionally, the method comprises assessing the one or more clinical manifestations of lupus by determining the difference between the cells and/or tissues of the subject prior to treatment administration compared to the cells and/or tissues of the subject after treatment administration.

The autoimmune disease of the present method may be Systemic Lupus Erythematosus (SLE). The subject of the present method may be a mammal, wherein the mammal is a mouse or a human. The antibody-producing cells of the present method may be B cells. One or more clinical manifestations of the autoimmune disease in the subject of the present method comprise an increase in B cells. Finally, the chimeric antigen receptor (CAR) of the present method comprises anti-CD19.

### BRIEF DESCRIPTION OF THE DRAWINGS

FIG. 1A is a schematic of a DNA construct (e.g., a retroviral cassette) for transducing murine cytotoxic T cells with a chimeric antigen receptor (CAR) comprising anti-CD19 or anti-CD138.

FIG. 1B is a schematic of the functional domains of the protein structure for the anti-CD19 CAR.

FIG. 1C is a schematic of the claimed method by which different treatment constructs are generated, including manipulation of retroviruses vs. lentiviruses, different promoters, T cell culture conditions, injection procedures, and different mouse models.

FIG. 1D is a contour plot showing blood lymphocyte levels of a non-irradiated mouse, which had comparable levels of CD19+ B cells and CD3+ T cells as the irradiated mouse at 18 days post-irradiation (see FIG. 1F).

FIG. 1E is a contour plot showing blood lymphocyte levels of a mouse that had reduced levels of CD19+ B cells and CD3+ T cells at 8 days after 5.2 Gy of irradiation.

FIG. 1F is a contour plot showing blood lymphocyte levels of the mouse of FIG. 1E, which recovered from transient myeloablation with 5.2 Gy of irradiation and had normal levels of CD19+ B cells and CD3+ T cells at 18 days post-irradiation.

FIG. 2A shows an 8401 bp plasmid map for recombinant vector pMSGV1-1D3-28Z 1-3 SV40-m comprising an 1D3 scFv, an anti-CD19 CAR, an mCherry gene, and a 5′ and a 3′ LTR sequence.

FIG. 2B shows the polynucleotide sequence alignment of the regulatory and coding domains of recombinant vector construct, pMSGV1-1D3-28Z 1-3 SV40-m, of FIG. 2A (SEQ ID NO: 1).

FIG. 2C is a protein sequence for one embodiment of the scFᵥ portion of a CAR of the present methods.

FIG. 3A is a table summarizing the viral vectors utilized in the claimed methods, such as A-MLV-CAR, G-MLV-CAR, and G-LV-CAR.

FIG. 3B is a schematic showing the first of three virus delivery systems or viral vectors expressing the CAR construct of the claimed method, including eco- and amphotropic retrovirus and G-pseudotyped lentivirus for delivery to cytotoxic T cells.

FIG. 3C is a schematic showing the second of three virus delivery systems or viral vectors expressing the CAR construct of the claimed method, including eco- and amphotropic retrovirus and G-pseudotyped lentivirus for delivery to cytotoxic T cells.

FIG. 3D is a schematic showing the third of three virus delivery systems or viral vectors expressing the CAR construct of the claimed method, including eco- and amphotropic retrovirus and G-pseudotyped lentivirus for delivery to cytotoxic T cells.

FIG. 3E is a scatter plot showing anti-CD3 (x-axis) and anti-Fab (y-axis) staining in CD8+ B cell lymphocytes that were not transduced (control) with cytotoxic T cells comprising an anti-CD19 CAR extracellular domain after 72 hours.

FIG. 3F is a scatter plot showing anti-CD3 (x-axis) and anti-Fab (y-axis) staining in CD8+ B cell lymphocytes transduced with cytotoxic T cells comprising a MSGV-1-G viral vector construct and a CAR extracellular domain after 72 hours.

FIG. 3G is a scatter plot showing anti-CD3 (x-axis) and anti-Fab (y-axis) staining in CD8+ B cell lymphocytes transduced with cytotoxic T cells comprising an Ampho-MSGV-1 viral vector construct and a CAR extracellular domain after 72 hours.

FIG. 3H is a scatter plot showing anti-CD3 (x-axis) and anti-Fab (y-axis) staining in CD8+ B cell lymphocytes transduced with cytotoxic T cells comprising an FEW-1D3 viral vector construct and a CAR extracellular domain after 72 hours.

FIG. 4A is a scatter plot showing that in NZBxNZW F1 female mice treated with the cytotoxic T cells comprising a vector construct and a CAR extracellular domain, B cells were depleted near background levels soon after treatment.

FIG. 4B is a scatter plot showing that in NZBxNZW F1 female mice treated with the cytotoxic T cells comprising a vector construct and a CAR extracellular domain, maintained B cells depletion near background levels soon over 6 months after treatment or until mice were sacrificed.

FIG. 5A is a graph showing approximately 93% CAR expression, identified by anti-Fab staining, in human Jurkat cancer cells.

FIG. 5B is a graph showing approximately 16% CAR expression, identified by anti-Fab staining, in primary, CD8+ T cells isolated from NZBxNZW F1 female mice.

FIG. 6A shows scatter plots for 10,000 cell events from each of 40 NZBxNZW F1 female mice equally separated into treatment groups comprising 9-10 mice each, and treated with one of the followings (from bottom to top): 1) no vector construct (i.e., negative control), 2) a G-LV-CAR vector construct, 3) a G-MLV-CAR vector construct, and 4) a A-MLV-CAR vector construct. The data indicates that depletion of B cells in the subject is stably maintained 11 weeks post-treatment.

FIG. 6B shows scatter plots for 10,000 cell events from a single NZBxNZW F1 female mouse (#9) that did not show a stable B cell depletion phenotype. While mouse #9 showed significant B cells depletion with only 1.996% of B cells present 4 weeks after cytotoxic T cell/CAR treatment, the B cells were observed to increase to 26.77% after 8 weeks, and to 46.93% after 12 weeks of treatment, while falling back down a bit to 30.54% by week 16. Mouse #9 was the only animal in the study to show this unstable B cell depletion phenotype.

FIG. 7A is a graph showing anti-sera IgG titers in NZBxNZW F1 female mice not treated with cytotoxic T cells comprising a vector construct and a CAR extracellular domain (negative control) after 3 months.

FIG. 7B is a graph showing anti-sera IgG titers in NZBxNZW F1 female mice treated with cytotoxic T cells comprising a vector construct and an a CAR extracellular domain remained at background levels after 3 months.

FIG. 8A is a graph showing anti-DNA titers in NZBxNZW F1 female mice plasma cells: 1) not treated with cytotoxic T cells comprising a vector construct and a CAR extracellular domain (negative control), 2) that are positive for B cells, and 3) that are negative for B cells.

FIG. 8B is a graph showing anti-IgG titers in NZBxNZW F1 female mice plasma cells: 1) not treated with cytotoxic T cells comprising a vector construct and a CAR extracellular domain (negative control), 2) that are positive for B cells, and 3) that are negative for B cells.

FIG. 9 is a graph showing observed changes in proteinuria of NZBxNZW F1 female mice: 1) prior to treatment with cytotoxic T cells comprising a vector construct and a CAR extracellular domain (negative control), 2) that are negative for B cells, and 3) that are positive for B cells.

FIG. 10A is an image of kidney cells from an untreated, control mouse stained with haematoxylin eosin (H & E) to show enlarged, hypercellular glomeruli.

FIG. 10B is an image of kidney cells from an untreated, control mouse stained red with Alexafluor 647 to show IgG deposits.

FIG. 10C is an image of H & E stained kidney cells from a mouse treated with cytotoxic T cells comprising a vector construct and a CAR extracellular domain showing normal appearing glomeruli as compared to the enlarged, hypercellular glomeruli of the untreated control mouse shown in FIG. 11A.

FIG. 10D is an image of kidney cells from a mouse treated with cytotoxic T cells comprising a vector construct and a CAR extracellular domain stained red with Alexafluor 647 to show a reduction of IgG deposits as compared to the IgG deposits of the untreated control mouse shown in FIG. 11B.

FIG. 10E is an image of an enlarged spleen from an untreated control mouse measuring approximately 3 cm as compared to the image of the normal-sized spleen measuring approximately 2 cm from a mouse treated with cytotoxic T cells comprising a vector construct and a CAR extracellular domain.

FIG. 11 is a graph showing the survival curve of untreated, control mice and mice treated with cytotoxic T cells comprising a vector construct and a CAR extracellular domain over the course of approximate one year.

FIG. 12 shows scatter plots for 10,000 cell events from each of 8 NZBxNZW F1 female mice that were separated into B cell depleted and B cell intact treatment groups comprising 1-3 mice each. B cells were removed from the mice, labeled with CSFE, and injected back into the recipient mouse. B cell activity was measured by assessing CSFE and CD19 cell expression at 1 hour and 5 days after injection with CFSE-labeled B cells. The data indicates that depletion of B cells in the subject is stably maintained for 7 months post-treatment.

FIG. 13A shows scatter plots for 10,000 cell events from each of 23 MRL/lpr female mice equally separated into two treatment groups comprising 11-12 mice each, and treated with one of the followings: 1) no vector construct (i.e., negative control) and 2) a CAR vector construct. The data indicates that depletion of B cells in 100% of the CAR-treated subjects was stably maintained indefinitely post-treatment.

FIG. 13B is a graph showing observed changes in proteinuria of MRL/lpr female mice: 1) at 3 weeks prior to treatment with cytotoxic T cells comprising a vector construct 2) CD19-deficient (CD19d) mice, and 3) CD19 sufficient (CD19s) mice.

FIG. 13C is a graph showing anti-IgG titers in MRL/lpr female mice plasma cells for: 1) an untreated, negative control and 2) after treatment with cytotoxic T cells comprising a vector construct and a CAR extracellular domain.

FIG. 13D is a graph showing anti-DNA titers in MRL/lpr female mice plasma cells for: 1) an untreated, negative control and 2) after treatment with cytotoxic T cells comprising a vector construct and a CAR extracellular domain.

FIG. 14 shows images of alopecia, skin lesions, and scabs on the ears and/or tails of untreated, control mice (bottom panel) as compared to mice treated with cytotoxic T cells comprising a vector construct and a CAR extracellular domain (top panel) showing no or very little alopecia (i.e., loss of hair) along the nose and face, skin lesions, and scabs on the ears and/or tails of the mice.

### DETAILED DESCRIPTION

A detailed description of the claimed methods and compositions of the present disclosure are described in detail by way of these clauses as follows.

1. A method of treating an autoimmune disease in a subject, the method comprising:
administering a plurality of engineered cytotoxic T cells to the subject, wherein each of the plurality of engineered cytotoxic T cells comprise a recombinant vector that expresses a chimeric antigen receptor,
depleting the number of antibody-producing cells in the subject,
improving one or more clinical manifestations of the autoimmune disease in the subject, and
treating the autoimmune disease in the subject.

2. The method of clause 1, wherein the autoimmune disease is Systemic Lupus Erythematosus (SLE).

3. The method of clause 1, wherein the autoimmune disease is rheumatoid arthritis.

4. The method of clause 1, wherein the autoimmune disease is diabetes.

5. The method of clause 4, wherein the diabetes is type 1 diabetes.

6. The method of clause 1, wherein the autoimmune disease is scleroderma.

7. The method of clause 1, wherein the autoimmune disease is Grave's Disease.

8. The method of clause 1, wherein the autoimmune disease is not cancer.

9. The method of clause 8, wherein the cancer is leukemia.

10. The method of clause 8, wherein the cancer is lymphoma

11. The method of any one of clauses 1-10, wherein the subject is female.

12. The method of any one of clauses 1-10, wherein the subject is a mammal.

13. The method of clause 12, wherein the mammal is a mouse.

14. The method of clause 13, wherein the mouse is a NZBxNZW mouse.

15. The method of clause 13, wherein the mouse is a not a C3H mouse.

16. The method of clause 13, wherein the mouse is a MRL/lpr mouse.

17. The method of clause 12, wherein the mammal is a human.

18. The method of any one of clauses 1-10, wherein the subject is a patient.

19. The method of clause 18, wherein the patient is a female patient.

20. The method of any one of clauses 1-19, wherein the one or more clinical manifestations of the autoimmune disease in the subject comprise proteinuria.

21. The method of any one of clauses 1-20, wherein the one or more clinical manifestations of the autoimmune disease in the subject comprise alopecia.

22. The method of clause 1, wherein the one or more clinical manifestations of the autoimmune disease in the subject comprises organ enlargement or enlargement of a portion of an organ.

23. The method of clause 22, wherein the organ is a spleen or a kidney.

24. The method of any one of clauses 1-23, wherein the one or more clinical manifestations of the autoimmune disease in the subject comprise hypercellular glomeruli.

25. The method of any one of clauses 1-24, wherein the one or more clinical manifestations of the autoimmune disease in the subject comprise IgG tissue deposits.

26. The method of any one of clauses 1-25, wherein the one or more clinical manifestations of the autoimmune disease in the subject comprise an increase in B cells.

27. The method of any one of clauses 1-26, wherein the one or more clinical manifestations of the autoimmune disease in the subject comprise skin lesions.

28. The method of any one of clauses 1-27, wherein the antibody-producing cells are B cells.

29. The method of clause 28, wherein the B cells are plasma cells. 30. The method of any one of clauses 1-29, wherein the plurality of engineered cytotoxic T cells are derived from the subject.

31. The method of clause 30, wherein the plurality of engineered cytotoxic T cells are murine cytotoxic T cells.

32. The method of clause 30, wherein the plurality of engineered cytotoxic T cells are human cytotoxic T cells.

33. The method of any one of clauses 1-32, wherein depletion of the antibody-producing cells in the subject is maintained indefinitely.

34. The method of any one of clauses 1-32, wherein the depletion of the antibody-producing cells in the subject is maintained for over 6 months.

35. The method of any one of clauses 1-32, wherein the depletion of the antibody-producing cells in the subject is maintained for at least 7 months.

36. The method of any one of clauses 1-32, wherein the depletion of the antibody-producing cells in the subject is maintained for at least 3 months.

37. The method of any one of clauses 1-32, wherein the depletion of the antibody-producing cells in the subject is maintained for at least 12 months.

38. The method of any one of clauses 1-37, wherein the chimeric antigen receptor (CAR) comprises anti-CD19.

39. The method of any one of clauses 1-37, wherein the chimeric antigen receptor (CAR) comprises anti-CD138

40. The method of any one of clauses 1-37, wherein the chimeric antigen receptor (CAR) comprises anti-BCMA

41. The method of any one of clauses 1-40, wherein the recombinant vector of each of the plurality of engineered cytotoxic T cells further comprises:
i. a promoter and
ii. a gene.

42. The method of clause 41, wherein the promoter comprises SV40.

43. The method of clause 41, wherein the promoter comprises PGK.

44. The method of clause 41, wherein the promoter comprises murine leukemia virus (MLV).

45. The method of clause 41, wherein the promoter comprises mouse stem cell virus (MSCV).

46. The method of clause 41, wherein the promoter comprises EF-1α.

47. The method of any one of clauses 41-46, wherein the gene is mCherry.

48. The method of any one of clauses 1-47, wherein the recombinant vector is an inducible vector.

49. The method of clause 48, wherein the inducible vector is induced by doxycycline.

50. The method of any one of clauses 1-49, wherein the recombinant vector further comprises long terminal repeats (LTRs).

51. The method of clause 50, wherein the long terminal repeats are 3′ LTRs.

52. The method of clause 50, wherein the long terminal repeats are 5′ LTRs.

53. The method of clause 50, wherein the long terminal repeats are 3′ LTRs and 5′ LTRs.

54. The method of any one of clauses 1-53, wherein the recombinant vector comprises SEQ ID NO: 1.

55. The method of any one of clauses 1-54, wherein the recombinant vector is a viral vector.

56. The method of clause 55, wherein the viral vector is A-MLV-CAR.

57. The method of clause 55, wherein the viral vector is G-MLV-CAR.

58. The method of clause 55, wherein the viral vector is G-LV-CAR.

59. The method of any one of clauses 1-58, wherein the recombinant vector further comprises a vector particle.

60. The method of clause 59, wherein the vector particle is a retrovirus.

61. The method of clause 59, wherein the vector particle is a lentivirus.

62. The method of clause 59, wherein the pseudotype of the vector particle is an amphotrophic envelope protein.

63. The method of clause 59, wherein the pseudotype of the vector particle is an envelope glycoprotein from vesicular stomatitis virus.

64. A method of treating one or more clinical manifestations of lupus in a subject, the method comprising:
administering a plurality of engineered cytotoxic T cells to the subject, wherein each of the plurality of engineered cytotoxic T cells comprise a recombinant vector that expresses a chimeric antigen receptor,
reducing or depleting the number of antibody-producing cells in the subject,
preventing, delaying, or reversing one or more clinical manifestations of lupus in the subject, and
treating the one or more clinical manifestations of lupus in the subject.

65. The method of clause 64, wherein the lupus is Systemic Lupus Erythematosus (SLE).

66. The method of clause 64, wherein the lupus is not cancer.

67. The method of clause 66, wherein the cancer is leukemia.

68. The method of clause 66, wherein the cancer is lymphoma

69. The method of any one of clauses 64-68, wherein the subject is female.

70. The method of any one of clauses 64-69, wherein the subject is a mammal.

71. The method of clause 70, wherein the mammal is a mouse.

72. The method of clause 71, wherein the mouse is a NZBxNZW mouse.

73. The method of clause 71, wherein the mouse is not a C3H mouse.

74. The method of clause 71, wherein the mouse is a MRL/lpr mouse.

75. The method of clause 70, wherein the mammal is a human.

76. The method of any one of clauses 64-75, wherein the subject is a patient.

77. The method of clause 76, wherein the patient is a female patient.

78. The method any one of clauses 64-77, wherein the one or more clinical manifestations of lupus in the subject comprises proteinuria.

79. The method of any one of clauses 64-78, wherein the one or more clinical manifestations of lupus the subject comprises alopecia.

80. The method of any one of clauses 64-79, wherein the one or more clinical manifestations of lupus in the subject comprises organ enlargement or enlargement of a portion of an organ.

81. The method of clause 80, wherein the organ is a spleen or a kidney.

82. The method of any one of clauses 64-81, wherein the one or more clinical manifestations of lupus in the subject comprises hypercellular glomeruli.

83. The method of any one of clauses 64-82, wherein the one or more clinical manifestations of lupus in the subject comprises IgG tissue deposits.

84. The method of any one of clauses 64-83, wherein the one or more clinical manifestations of lupus in the subject comprises an increase in B cells.

85. The method of any one of clauses 64-84, wherein the one or more clinical manifestations of lupus in the subject comprises skin lesions.

86. The method of any one of clauses 64-85, wherein the antibody-producing cells are B cells.

87. The method of clause 86, wherein the B cells are plasma cells.

88. The method of any one of clauses 64-87, wherein the plurality of engineered cytotoxic T cells are derived from the subject.

89. The method of clause 88, wherein the plurality of engineered cytotoxic T cells are murine cytotoxic T cells.

90. The method of clause 88, wherein the plurality of engineered cytotoxic T cells are human cytotoxic T cells.

91. The method of any one of clauses 64-90, wherein depletion of the antibody-producing cells in the subject is maintained indefinitely.

92. The method of any one of clauses 64-90, wherein the depletion of the antibody-producing cells in the subject is maintained for over 6 months.

93. The method of any one of clauses 64-90, wherein the depletion of the antibody-producing cells in the subject is maintained for at least 7 months.

94. The method of any one of clauses 64-90, wherein the depletion of the antibody-producing cells in the subject is maintained for at least 3 months.

95. The method of any one of clauses 64-90, wherein the depletion of the antibody-producing cells in the subject is maintained for at least 12 months.

96. The method of any one of clauses 64-95, wherein the chimeric antigen receptor (CAR) comprises anti-CD19.

97. The method of any one of clauses 64-95, wherein the chimeric antigen receptor (CAR) comprises anti-CD138.

98. The method of any one of clauses 64-95, wherein the chimeric antigen receptor (CAR) comprises anti-BCMA

99. The method of any one of clauses 64-98, wherein the recombinant vector of each of the plurality of engineered cytotoxic T cells further comprises:
iii. a promoter and
iv. a gene.

100. The method of clause 99, wherein the promoter comprises SV40.

101. The method of clause 99, wherein the promoter comprises PGK.

102. The method of clause 99, wherein the promoter comprises murine leukemia virus (MLV).

103. The method of clause 99, wherein the promoter comprises mouse stem cell virus (MSCV).

104. The method of clause 99, wherein the promoter comprises EF-1α.

105. The method of any one of clauses 99-104, wherein the gene is mCherry.

106. The method of any one of clauses 64-105, wherein the recombinant vector is an inducible vector.

107. The method of clause 106, wherein the inducible vector is induced by doxycycline.

108. The method of any one of clauses 64-107, wherein the recombinant vector further comprises long terminal repeats (LTRs).

109. The method of clause 108, wherein the long terminal repeats are 3′ LTRs.

110. The method of clause 108, wherein the long terminal repeats are 5′ LTRs.

111. The method of clause 108, wherein the long terminal repeats are 3′ LTRs and 5′ LTRs.

112. The method of any one of clauses 64-111, wherein the recombinant vector comprises SEQ ID NO: 1.

113. The method of any one of clauses 64-112, wherein the recombinant vector is a viral vector.

114. The method of clause 113, wherein the viral vector is A-MLV-CAR.

115. The method of clause 113, wherein the viral vector is G-MLV-CAR. 116. The method of clause 113, wherein the viral vector is G-LV-CAR.

117. The method of any one of clauses 64-116, wherein the recombinant vector further comprises a vector particle.

118. The method of clause 117, wherein the vector particle is a retrovirus.

119. The method of clause 117, wherein the vector particle is a lentivirus.

120. The method of clause 117, wherein the pseudotype of the vector particle is an amphotrophic envelope protein.

121. The method of clause 117, wherein the pseudotype of the vector particle is an envelope glycoprotein from vesicular stomatitis virus.

122. A method of reducing or depleting the number of antibody-producing cells in a subject, the method comprising:
administering a plurality of engineered cytotoxic T cells to the subject, wherein each of the plurality of engineered cytotoxic T cells comprise a recombinant vector that expresses a chimeric antigen receptor, and
reducing or depleting the number of antibody-producing cells in the subject.

123. The method of clause 122, wherein the subject is female.

124. The method of clause 122, wherein the subject is a mammal.

125. The method of clause 122, wherein the mammal is a mouse.

126. The method of clause 125, wherein the mouse is a NZBxNZW mouse.

127. The method of clause 125, wherein the mouse not is a C3H mouse.

128. The method of clause 125, wherein the mouse is a MRL/lpr mouse.

129. The method of clause 122, wherein the mammal is a human.

130. The method of any one of clauses 122-129, wherein the subject is a patient.

131. The method of clause 130, wherein the patient is a female patient.

132. The method of any one of clauses 122-131, wherein the one or more clinical manifestations of the autoimmune disease in the subject comprises proteinuria.

133. The method of any one of clauses 122-132, wherein the antibody-producing cells are B cells.

134. The method of clause 133, wherein the B cells are plasma cells.

135. The method of any one of clauses 122-134, wherein the plurality of engineered cytotoxic T cells are derived from the subject.

136. The method of clause 135, wherein the plurality of engineered cytotoxic T cells are murine cytotoxic T cells.

137. The method of clause 135, wherein the plurality of engineered cytotoxic T cells are human cytotoxic T cells.

138. The method of any one of clauses 122-137, wherein the reduction or depletion of the antibody-producing cells in the subject is maintained indefinitely.

139. The method of any one of clauses 122-137, wherein the reduction or depletion of the antibody-producing cells in the subject is maintained for over 6 months.

140. The method of any one of clauses 122-137, wherein the reduction or depletion of the antibody-producing cells in the subject is maintained for at least 7 months.

141. The method of any one of clauses 122-137, wherein the reduction or depletion of the antibody-producing cells in the subject is maintained for at least 3 months.

142. The method of any one of clauses 122-137, wherein the reduction or depletion of the antibody-producing cells in the subject is maintained for at least 12 months.

143. The method of any one of clauses 122-142, wherein the chimeric antigen receptor (CAR) comprises anti-CD19.

144. The method of any one of clauses 122-142, wherein the chimeric antigen receptor (CAR) comprises anti-CD138

145. The method of any one of clauses 122-142, wherein the chimeric antigen receptor (CAR) comprises anti-BCMA

146. The method of clause 122, wherein the recombinant vector of each of the plurality of engineered cytotoxic T cells further comprises:
v. a promoter and
vi. a gene.

147. The method of clause 146, wherein the promoter comprises SV40.

148. The method of clause 146, wherein the promoter comprises PGK.

149. The method of clause 146, wherein the promoter comprises murine leukemia virus (MLV).

150. The method of clause 146, wherein the promoter comprises mouse stem cell virus (MSCV).

151. The method of clause 146, wherein the promoter comprises EF-1a.

152. The method of any one of clauses 122-151, wherein the gene is mCherry.

153. The method of any one of clauses 122-152, wherein the recombinant vector is an inducible vector.

154. The method of clause 153, wherein the inducible vector is induced by doxycycline.

155. The method of any one of clauses 122-154, wherein the recombinant vector further comprises long terminal repeats (LTRs).

156. The method of clause 155, wherein the long terminal repeats are 3′ LTRs.

157. The method of clause 155, wherein the long terminal repeats are 5′ LTRs. 158. The method of clause 155, wherein the long terminal repeats are 3′ LTRs and 5′ LTRs.

159. The method of any one of clauses 122-158, wherein the recombinant vector comprises SEQ ID NO: 1.

160. The method of any one of clauses 122-159, wherein the recombinant vector is a viral vector.

161. The method of clause 160, wherein the viral vector is A-MLV-CAR.

162. The method of clause 160, wherein the viral vector is G-MLV-CAR.

163. The method of clause 160, wherein the viral vector is G-LV-CAR.

164. The method of any one of clauses 122-163, wherein the recombinant vector further comprises a vector particle.

165. The method of clause 164, wherein the vector particle is a retrovirus.

166. The method of clause 164, wherein the vector particle is a lentivirus.

167. The method of clause 164, wherein the pseudotype of the vector particle is an amphotrophic envelope protein.

168. The method of clause 164, wherein the pseudotype of the vector particle is an envelope glycoprotein from vesicular stomatitis virus.

169. A method of monitoring efficacy of lupus treatment in a subject, the method comprising:
measuring one or more clinical manifestations of lupus in the cells and/or tissues of a subject prior to treatment administration,
administering a treatment construct comprising a plurality of engineered cytotoxic T cells to the subject,
   wherein each of the plurality of engineered cytotoxic T cells comprise a recombinant vector that expresses a chimeric antigen receptor,
at least one hour after treatment administration, remeasuring the one or more clinical manifestations of lupus in the subject,
assessing the one or more clinical manifestations of lupus by determining the difference between the cells and/or tissues of the subject prior to treatment administration compared to the cells and/or tissues of the subject after treatment administration.

170. The method of clause 169, wherein the lupus is Systemic Lupus Erythematosus (SLE).

171. The method of clause 169, wherein the lupus is not cancer.

172. The method of clause 171, wherein the cancer is leukemia.

173. The method of clause 171, wherein the cancer is lymphoma

174. The method of clause 169, wherein the subject is female.

175. The method of clause 169, wherein the subject is a mammal.

176. The method of clause 175, wherein the mammal is a mouse.

177. The method of clause 176, wherein the mouse is a NZBxNZW mouse.

178. The method of clause 176, wherein the mouse is not a C3H mouse.

179. The method of clause 176, wherein the mouse is a MRL/lpr mouse.

180. The method of clause 175, wherein the mammal is a human.

181. The method of any one of clauses 169-180, wherein the subject is a patient.

182. The method of clause 181, wherein the patient is a female patient.

183. The method of any one of clauses 169-182, wherein the one or more clinical manifestations of lupus in the subject comprises proteinuria.

184. The method of any one of clauses 169-183, wherein the one or more clinical manifestations of lupus in the subject comprises alopecia.

185. The method of any one of clauses 169-184, wherein the one or more clinical manifestations of lupus in the subject comprises organ enlargement or enlargement of a portion of an organ.

186. The method of clause 185, wherein the organ is a spleen or a kidney.

187. The method of any one of clauses 169-186, wherein the one or more clinical manifestations of lupus in the subject comprises hypercellular glomeruli.

188. The method of any one of clauses 169-187, wherein the one or more clinical manifestations of lupus in the subject comprises IgG tissue deposits.

189. The method of any one of clauses 169-188, wherein the one or more clinical manifestations of lupus in the subject comprises an increase in B cells.

190. The method of any one of clauses 169-189, wherein the one or more clinical manifestations of lupus in the subject comprises skin lesions.

191. The method of clause 189, wherein the B cells are plasma cells.

192. The method of any one of clauses 169-191, wherein the plurality of engineered cytotoxic T cells are derived from the subject.

193. The method of clause 193, wherein the plurality of engineered cytotoxic T cells are murine cytotoxic T cells.

194. The method of clause 193, wherein the plurality of engineered cytotoxic T cells are human cytotoxic T cells.

195. The method of clause 189, wherein assessing the increase in B cells in the subject prior to treatment administration compared to the number of B cells of the subject after treatment administration shows a reduction or depletion of the B cells in the subject that is maintained indefinitely.

196. The method of clause 189, wherein assessing the increase in B cells in the subject prior to treatment administration compared to the number of B cells of the subject after treatment administration shows a reduction or depletion of the B cells in the subject that is maintained for over 6 months.

197. The method of clause 189, wherein assessing the increase in B cells in the subject prior to treatment administration compared to the number of B cells of the subject after treatment administration shows a reduction or depletion of the B cells in the subject that is maintained for at least 7 months.

198. The method of clause 189, wherein assessing the increase in B cells in the subject prior to treatment administration compared to the number of B cells of the subject after treatment administration shows a reduction or depletion of the B cells in the subject that is maintained for at least 3 months.

199. The method of clause 189, wherein assessing the increase in B cells in the subject prior to treatment administration compared to the number of B cells of the subject after treatment administration shows a reduction or depletion of the B cells in the subject that is maintained for at least 12 months.

200. The method of any one of clauses 169-199, wherein the chimeric antigen receptor (CAR) comprises anti-CD19.

201. The method of any one of clauses 169-199, wherein the chimeric antigen receptor (CAR) comprises anti-CD138.

202. The method of any one of clauses 169-199, wherein the chimeric antigen receptor (CAR) comprises anti-BCMA.

203. The method of any one of clauses 169-199, wherein the recombinant vector of each of the plurality of engineered cytotoxic T cells further comprises:
vii. a promoter and
viii. a gene.

204. The method of clause 203, wherein the promoter comprises SV40.

205. The method of clause 203, wherein the promoter comprises PGK.

206. The method of clause 203, wherein the promoter comprises murine leukemia virus (MLV).

207. The method of clause 203, wherein the promoter comprises mouse stem cell virus (MSCV).

208. The method of clause 203, wherein the promoter comprises EF-1α.

209. The method of any one of clauses 203-208, wherein the gene is mCherry.

210. The method of any one of clauses 169-209, wherein the recombinant vector is an inducible vector.

211. The method of clause 210, wherein the inducible vector is induced by doxycycline.

212. The method of any one of clauses 169-211, wherein the recombinant vector further comprises long terminal repeats (LTRs).

213. The method of clause 212, wherein the long terminal repeats are 3′ LTRs.

214. The method of clause 212, wherein the long terminal repeats are 5′ LTRs.

215. The method of clause 212, wherein the long terminal repeats are 3′ LTRs and 5′ LTRs.

216. The method of any one of clauses 169-215, wherein the recombinant vector comprises SEQ ID NO: 1.

217. The method of any one of clauses 169-216, wherein the recombinant vector is a viral vector.

218. The method of clause 217, wherein the viral vector is A-MLV-CAR.

219. The method of clause 217, wherein the viral vector is G-MLV-CAR.

220. The method of clause 217, wherein the viral vector is G-LV-CAR.

221. The method of any one of clauses 169-220, wherein the recombinant vector further comprises a vector particle.

222. The method of clause 221, wherein the vector particle is a retrovirus.

223. The method of clause 221, wherein the vector particle is a lentivirus.

224. The method of clause 221, wherein the pseudotype of the vector particle is an amphotrophic envelope protein.

225. The method of clause 221, wherein the pseudotype of the vector particle is an envelope glycoprotein from vesicular stomatitis virus.

As used herein, the articles, “a”, “an”, and “the” are used herein to refer to one or to more than one (i.e., to at least one) of the grammatical object of the article unless the context clearly and unambiguously dictates otherwise. By way of example, “an element” means one element or more than one element.

As used herein, the term “adjuvant” refers to a substance that elicits an enhanced immune response when used in combination with a specific antigen.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

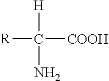

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an amino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

As used herein, an “analog” of a chemical compound is a compound that, by way of example, resembles another in structure but is not necessarily an isomer (e.g., 5-fluorouracil is an analog of thymine).

The term “antibody,” as used herein, refers to an immunoglobulin molecule which is able to specifically bind to a specific epitope on an antigen. Antibodies may be intact immunoglobulins derived from natural sources or from recombinant sources and may be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ, complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ may be reduced under mild conditions to break the disulfide linkage in the hinge region, thereby converting the F(ab′)₂ dimer into an Fab₁ monomer. The Fab₁ monomer is essentially an Fab with part of the hinge region. While various antibody fragments are defined in terms of the digestion of an intact antibody, one of ordinary skill in the art will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

The term “single chain antibody” refers to an antibody wherein the genetic information encoding the functional fragments of the antibody is located in a single contiguous length of DNA.

The term “humanized” refers to an antibody wherein the constant regions have at least about 80% or greater homology to human immunoglobulin. Additionally, some of the nonhuman, such as murine, variable region amino acid residues may be modified to contain amino acid residues of human origin.

Humanized antibodies have been referred to as “reshaped” antibodies. Manipulation of the complementarity-determining regions (CDR) is a way of achieving humanized antibodies as described in Jones, et al., Nature 321:522 (1988), Riechmann, et al., Nature 332:323 (1988), and Winter & Milstein, Nature 349:293 (1991).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen may be derived from organisms, subunits of proteins/antigens, killed or inactivated whole cells or lysates.

“Antimicrobial” as used herein, includes antibacterial, antifungal, and antiviral agents.

An “autoimmune disease,” as used herein, is a chronic disease conditions in which the immune system escapes regulation by self-tolerance and attacks normal host tissues and cellular functions in ways that generate typical and distinguishing signs of autoimmunity known in the art (i.e., clinical manifestations or symptoms). In many autoimmune diseases, the pathogenic mechanisms require B cell production of autoantibodies. Autoantibodies are produced by B cells and bind to substances, molecules, and/or cell surfaces that are part of a normal healthy subject, organism, or patient. The binding of antibodies or autoantibodies, such as those produced by B cells, directly or through activation of additional immune functions, leads to lasting and progressive tissue damage to the subject.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full-length protein that are capable of specific binding to their natural ligand or of performing the function of the protein.

The term “biological sample,” as used herein, refers to samples obtained from a subject, including, but not limited to, sputum, mucus, phlegm, tissues, biopsies, cerebrospinal fluid, blood, serum, plasma, other blood components, gastric aspirates, throat swabs, pleural effusion, peritoneal fluid, follicular fluid, ascites, skin, hair, tissue, blood, plasma, cells (e.g., B cells and/or plasma cells), saliva, sweat, tears, semen, stools, Pap smears, and urine. One of skill in the art will understand the type of sample needed.

A “biomarker” or “marker” is a specific biochemical in the body which has a particular molecular feature that makes it useful for measuring the progress of disease or the effects of treatment, or for measuring a process of interest.

The term “cancer”, as used herein, is defined as proliferation of cells whose unique trait (loss of normal controls) results in unregulated growth, lack of differentiation, local tissue invasion, and metastasis. Examples include but are not limited to, melanoma, leukemia, lymphoma, breast cancer, prostate cancer, ovarian cancer, uterine cancer, cervical cancer, skin cancer, pancreatic cancer, colorectal cancer, renal cancer and lung cancer.

As used herein, the term “carrier molecule” refers to any molecule that is chemically conjugated to a molecule of interest.

The term “cell surface protein” means a protein found where at least part of the protein is exposed at the outer aspect of the cell membrane. Examples include growth factor receptors. Illustrative examples include the chimeric antigen receptors or CARs of the present disclosure.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

As used herein, a “derivative” refers to a chemical compound that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, in one embodiment, the term “diagnosis” refers to detecting aberrant ALCAM expression due to cancers expressing ALCAM. In any method of diagnosis exists false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, may be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

As used herein, the term “expression” refers to the process by which a polynucleotide is transcribed into mRNA (including small RNA molecules) and/or the process by which the transcribed mRNA (also referred to as “transcript”) is subsequently translated into peptides, polypeptides, or proteins. Gene expression may be influenced by external signals, for example, exposure of a cell, tissue, or organism to an agent that increases or decreases gene expression. Expression of a gene may also be regulated anywhere in the pathway from DNA to RNA to protein. Regulation of gene expression occurs, for example, through controls acting on transcription, translation, RNA transport and processing, degradation of intermediary molecules, such as mRNA, or through activation, inactivation, compartmentalization, or degradation of specific protein molecules after they have been made, or by combinations thereof. Gene expression may be measured at the RNA level or the protein level by any method known in the art, including, without limitation, Northern blot, RT-PCR, Western blot, or in vitro, in situ, or in vivo protein activity assay(s).

As used herein, the term

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, a “functional” component is a component in a form in which it exhibits a property by which it is characterized. For example, a functional enzyme is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology.

As used herein, “homology” is used synonymously with “identity.”

The determination of percent identity between two nucleotide or amino acid sequences may be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is also incorporated into the NBLAST and XBLAST programs of Altschul, et al. (1990, J. Mol. Biol. 215:403-410), and may be accessed, for example at the National Center for Biotechnology Information (NCBI) world wide web site having the universal resource locator using the BLAST tool at the NCBI website. BLAST nucleotide searches may be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches may be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST may be utilized as described in Altschul et al. (1997, Nucleic Acids Res. 25:3389-3402). Alternatively, PSI-Blast or PHI-Blast may be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) may be used.

The percent identity between two sequences may be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “inhibit,” as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block.”

As used herein, an “instructional material” includes a publication, a recording, a diagram, or any other medium of expression which may be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

As used herein, the term “intron” refers to any nucleic acid sequence comprised in a gene (or expressed nucleotide sequence of interest) that is transcribed but not translated. Introns include untranslated nucleic acid sequence within an expressed sequence of DNA, as well as a corresponding sequence in RNA molecules transcribed therefrom.

A “construct” described herein may also contain sequences that enhance translation and/or mRNA stability, such as introns. Introns may be used in combination with a promoter sequence to enhance translation and/or mRNA stability.

As used herein, the terms “5′-untranslated region” or “5′-UTR” refers to an untranslated segment in the 5′ terminus of pre-mRNAs or mature mRNAs. For example, on mature mRNAs, a 5′-UTR typically harbors on its 5′ end a 7-methylguanosine cap and is involved in many processes such as splicing, polyadenylation, mRNA export towards the cytoplasm, identification of the 5′ end of the mRNA by the translational machinery, and protection of the mRNAs against degradation.

As used herein, the term “3′-untranslated region” or “3′-UTR” refers to an untranslated segment in a 3′ terminus of the pre-mRNAs or mature mRNAs. For example, on mature mRNAs this region harbors the poly-(A) tail and is known to have many roles in mRNA stability, translation initiation, and mRNA export.

As used herein, the term “isolated” refers to a biological component (including a nucleic acid or protein) that has been separated from other biological components in the cell of the organism in which the component naturally occurs (i.e., other chromosomal and extra-chromosomal DNA).

For example, an “isolated protein” refers to an amino acid sequence peptide, segment, or fragment which has been separated from sequences which flank it in a naturally occurring state, such as the amino acid sequences adjacent to the fragment in a genome in which it naturally occurs. The term also applies to proteins which have been substantially purified from other components which naturally accompany the protein, e.g., RNA or DNA which naturally accompany it in the cell. The term therefore includes, for example, a recombinant protein which is incorporated into a vector, into an autonomously replicating plasmid or virus, or into the genome of a prokaryote or eukaryote, or which exists as a separate molecule (e.g., as a synthetic, recombinant, chimeric, etc. protein) independent of other sequences. It also includes a recombinant protein which is part of a hybrid gene encoding additional polypeptide sequence.

A “ligand” is a compound that specifically binds to a target receptor.

A “receptor” is a compound that specifically binds to a ligand.

A ligand or a receptor (e.g., an antibody) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988, Antibodies, A Laboratory Manual, Cold Spring Harbor Publications, New York) for a description of immunoassay formats and conditions that may be used to determine specific immunoreactivity of the antibodies of the present disclosure.

As used herein, the term “linkage” refers to a connection between two groups. The connection may be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

The term “measuring the level of expression” or “determining the level of expression” as used herein refers to any measure or assay which may be used to correlate the results of the assay with the level of expression of a gene or protein of interest. Such assays include measuring the level of mRNA, protein levels, etc. and may be performed by assays such as northern and western blot analyses, binding assays, immunoblots, etc. The level of expression can include rates of expression and may be measured in terms of the actual amount of an mRNA or protein present. Such assays are coupled with processes or systems to store and process information and to help quantify levels, signals, etc. and to digitize the information for use in comparing levels.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the terms “nucleic acid molecule,” “nucleic acid,” or “polynucleotide” (all three terms being synonymous with one another) refer to a polymeric form of nucleotides, which may include both sense and anti-sense strands of RNA, cDNA, genomic DNA, and synthetic forms, and mixed polymers thereof. A “nucleotide” may refer to a ribonucleotide, deoxyribonucleotide, or a modified form of either type of nucleotide. A nucleic acid molecule is usually at least ten bases in length, unless otherwise specified. The terms may refer to a molecule of RNA or DNA of indeterminate length. The terms include single- and double-stranded forms of DNA. A nucleic acid molecule may include either or both naturally-occurring and modified nucleotides linked together by naturally occurring and/or non-naturally occurring nucleotide linkages.

Nucleic acid molecules may be modified chemically or biochemically, or may contain non-natural or derivatized nucleotide bases, as will be readily appreciated by those of ordinary skill in the art. Such modifications include, for example, labels, methylation, substitution of one or more of the naturally-occurring nucleotides with an analog, internucleotide modifications (e.g., uncharged linkages, such as, methyl phosphonates, phosphotriesters, phosphoramidates, carbamates, etc.; charged linkages, such as, phosphorothioates, phosphorodithioates, etc.; pendent moieties, such as, peptides; intercalators, such as, acridine, psoralen, etc.; chelators; alkylators; and modified linkages, such as, alpha anomeric nucleic acids, etc.). The term “nucleic acid molecule” also includes any topological conformation, including single-stranded, double-stranded, partially duplexed, triplexed, hairpinned, circular, and padlocked conformations.

Transcription proceeds in a 5′ to 3′ manner along a DNA strand. This means that RNA is made by sequential addition of ribonucleotide-5′-triphosphates to the 3′ terminus of the growing chain with a requisite elimination of the pyrophosphate. In either a linear or circular nucleic acid molecule, discrete elements (e.g., particular nucleotide sequences) may be referred to as being “upstream” relative to a further element if they are bonded or would be bonded to the same nucleic acid in the 5′ direction from that element. Similarly, discrete elements may be referred to as being “downstream” relative to a further element if they are or would be bonded to the same nucleic acid in the 3′ direction from that element.

As used herein, the term “base position” refers to the location of a given base or nucleotide residue within a designated nucleic acid. A designated nucleic acid may be defined by alignment with a reference nucleic acid.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Oligonucleotides may be formed by cleavage of longer nucleic acid segments or by polymerizing individual nucleotide precursors. Automated synthesizers allow the synthesis of oligonucleotides up to several hundred base pairs in length. Because oligonucleotides may bind to a complementary nucleotide sequence, they may be used as probes for detecting DNA or RNA. Oligonucleotides composed of DNA (oligodeoxyribonucleotides) may be used in Polymerase Chain Reaction, a technique for the amplification of small DNA sequences. In Polymerase Chain Reaction, an oligonucleotide is typically referred to as a “primer” which allows a DNA polymerase to extend the oligonucleotide and replicate the complementary strand.

As used herein, the term “operably linked” refers to a nucleic acid placed into a functional relationship with another nucleic acid. Generally, “operably linked” may mean that linked nucleic acids are contiguous. Linking may be accomplished by ligation at convenient restriction sites. If such sites do not exist, synthetic oligonucleotide adaptors or linkers are ligated or annealed to the nucleic acid and used to link the contiguous polynucleotide fragment. However, elements need not be contiguous to be operably linked.

By describing two polynucleotides as “operably linked,” it is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides or to peptides shorter than the full length native or mature protein.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative may be combined and which, following the combination, may be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

A “plurality” means at least two, and may comprise many, such as hundreds, thousands, or millions and that which are too innumerable to specifically quantify.

As used herein, the terms “Polymerase Chain Reaction” or “PCR” refer to a procedure or technique in which minute amounts of nucleic acid, RNA, and/or DNA, are amplified as described in U.S. Pat. No. 4,683,195. Generally, sequence information from the ends of the region of interest or beyond needs to be available, such that oligonucleotide primers may be designed. PCR primers will be identical or similar in sequence to opposite strands of the nucleic acid template to be amplified. The 5′ terminal nucleotides of the two primers may coincide with the ends of the amplified material. PCR may be used to amplify specific RNA sequences or DNA sequences from total genomic DNA and cDNA transcribed from total cellular RNA, bacteriophage, or plasmid sequences, etc. See generally Mullis et al., Cold Spring Harbor Symp. Quant. Biol., 51:263 (1987); Erlich, ed., PCR Technology, (Stockton Press, N Y, 1989).

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” mean a non-naturally occurring peptide or polypeptide. Synthetic peptides or polypeptides may be synthesized, for example, using an automated polypeptide synthesizer. Various solid phase peptide synthesis methods are known to those of skill in the art.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers may be labeled with, e.g., chromogenic, radioactive, or fluorescent moieties and used as detectable moieties.

Additionally, the term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates (i.e., A, T, G, and C) and at least one polymerization-inducing agent or enzyme such as Reverse Transcriptase or DNA polymerase. These reagents are typically present in a suitable buffer that may include constituents which are co-factors or which affect conditions, such as pH and the like at various suitable temperatures. A primer is preferably a single strand sequence, such that amplification efficiency is optimized, but double stranded sequences may be utilized.

As used herein, the term “probe” refers to an oligonucleotide or polynucleotide sequence that hybridizes to a target sequence. In the TaqMan® or TaqMan®-style assay procedure, the probe hybridizes to a portion of the target situated between the annealing site of the two primers. A probe includes about eight nucleotides, about ten nucleotides, about fifteen nucleotides, about twenty nucleotides, about thirty nucleotides, about forty nucleotides, or about fifty nucleotides. In some embodiments, a probe includes from about eight nucleotides to about fifteen nucleotides.

In the Southern blot assay procedure, the probe hybridizes to a DNA fragment that is attached to a membrane. A probe includes about ten nucleotides, about 100 nucleotides, about 250 nucleotides, about 500 nucleotides, about 1,000 nucleotides, about 2,500 nucleotides, or about 5,000 nucleotides. In some embodiments, a probe includes from about 500 nucleotides to about 2,500 nucleotides.

A probe may further include a detectable label, such as, a radioactive label, a biotinylated label, a fluorophore (e.g., Texas-Red®, fluorescein isothiocyanate, etc.). The detectable label may be covalently attached directly to the probe oligonucleotide, such that the label is located at the 5′ end or 3′ end of the probe. A probe comprising a fluorophore may also further include a quencher dye (e.g., Black Hole Quencher™, Iowa Black™, etc.).

As used herein, the terms “sequence identity” or “identity” may be used interchangeably and refer to nucleic acid residues in two sequences that are the same when aligned for maximum correspondence over a specified comparison window.

As used herein, the term “percentage of sequence identity” or “percentage of sequence homology” refers to a value determined by comparing two optimally aligned sequences (e.g., nucleic acid sequences or amino acid sequences) over a comparison window, wherein the portion of a sequence in the comparison window may comprise additions, substitutions, mismatches, and/or deletions (i.e., gaps) as compared to a reference sequence in order to obtain optimal alignment of the two sequences. A percentage is calculated by determining the number of positions at which an identical nucleic acid or amino acid residue occurs in both sequences to yield the number of matched positions, dividing the number of matched positions by the total number of positions in the comparison window, and multiplying the result by 100 to yield the percentage of sequence identity. Methods for aligning sequences for comparison are well known. Various bioinformatics or computer programs and alignment algorithms, such as ClustalW and Sequencher, are also well known in the art and/or described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50.

The National Center for Biotechnology Information (NCBI) Basic Local Alignment Search Tool (BLAST™; Altschul et al. (1990) J. Mol. Biol. 215:403-10) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method.

As used herein, the term “promoter” refers to a region of DNA that is generally located upstream of a gene (i.e., towards the 5′ end of a gene) and is necessary to initiate and drive transcription of the gene. A promoter may permit proper activation or repression of a gene that it controls. A promoter may contain specific sequences that are recognized by transcription factors. These factors may bind to a promoter DNA sequence, which results in the recruitment of RNA polymerase, an enzyme that synthesizes RNA from the coding region of the gene. The promoter generally refers to all gene regulatory elements located upstream of the gene, including, 5′-UTR, introns, and leader sequences.

As used herein, the term “upstream-promoter” refers to a contiguous polynucleotide sequence that is sufficient to direct initiation of transcription. As used herein, an upstream-promoter encompasses the site of initiation of transcription with several sequence motifs, which include a TATA Box, initiator (Intr) sequence, TFIIB recognition elements (BRE), and other promoter motifs (Jennifer, E. F. et al, (2002) Genes & Dev., 16: 2583-2592). The upstream-promoter provides the site of action to RNA polymerase II, a multi-subunit enzyme with the basal or general transcription factors like, TFIIA, B, D, E, F, and H. These factors assemble into a transcription pre-initiation complex (PIC) that catalyzes the synthesis of RNA from a DNA template.

The activation of the upstream-promoter is performed by the addition of regulatory DNA sequence elements to which various proteins bind and subsequently interact with the transcription initiation complex to activate gene expression. These gene regulatory element sequences interact with specific DNA-binding factors. These sequence motifs may sometimes be referred to as cis-elements. Such cis-elements, to which tissue-specific or development-specific transcription factors bind, individually or in combination, may determine the spatiotemporal expression pattern of a promoter at the transcriptional level. These cis-elements vary widely in the type of control they exert on operably linked genes. Some elements act to increase the transcription of operably-linked genes in response to environmental responses (e.g., temperature, moisture, and wounding). Other cis-elements may respond to developmental cues (e.g., germination, seed maturation, and flowering) or to spatial information (e.g., tissue specificity). See, for example, Langridge et al. (1989) Proc. Natl. Acad. Sci. USA 86:3219-23. These cis-elements are located at a varying distance from the transcription start point. Some cis-elements (called proximal elements) are adjacent to a minimal core promoter region, while other elements may be positioned several kilobases 5′ upstream or 3′ downstream of the promoter (enhancers).

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups of the present application.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” in reference to nucleic acid molecules does not require absolute purity (such as a homogeneous preparation). Instead, “purified” represents an indication that the sequence is relatively more pure than in its native cellular environment. For example, the “purified” level of nucleic acids should be at least 2-5 fold greater in terms of concentration or gene expression levels as compared to its natural level.

For example, a “highly purified” compound as used herein refers to a compound that is greater than 90% pure. A “significant detectable level” is an amount of contaminate that would be visible in the presented data and would need to be addressed/explained during analysis of the forensic evidence.

The claimed DNA molecules may be obtained directly from total DNA or from total RNA. In addition, cDNA clones are not naturally occurring, but rather are preferably obtained via manipulation of a partially purified, naturally occurring substance (messenger RNA). The construction of a cDNA library from mRNA involves the creation of a synthetic substance (cDNA). Individual cDNA clones may be purified from the synthetic library by clonal selection of the cells carrying the cDNA library. Thus, the process which includes the construction of a cDNA library from mRNA and purification of distinct cDNA clones yields an approximately 10⁶-fold purification of the native message. Likewise, a promoter DNA sequence may be cloned into a plasmid. Such a clone is not naturally occurring, but rather is preferably obtained via manipulation of a partially purified, naturally occurring substance, such as a genomic DNA library. Thus, purification of at least one order of magnitude, preferably two or three orders, and more preferably four or five orders of magnitude, is favored in these techniques.

Similarly, purification represents an indication that a chemical or functional change in the component DNA sequence has occurred. Nucleic acid molecules and proteins that have been “purified” include nucleic acid molecules and proteins purified by standard purification methods. The term “purified” also embraces nucleic acids and proteins prepared by recombinant DNA methods in a host cell or tissue, as well as chemically-synthesized nucleic acid molecules, proteins, and peptides.

A “receptor,” as used herein, is a compound that directly binds to a ligand. Many receptors are cell surface proteins that recognize signals from the exterior of the cell and transduce the signal to the interior of the cell to cause downstream effects and/or functional changes within the cell. Depending on the cell type, different cells may express different and/or different types of cell surface receptors. For example, most B cells uniquely express a surface receptor called CD19. Mature B cells that actively produce and secrete antibodies or autoantibodies also express the surface receptor, B cell maturation antigen (BCMA), and CD138. Therefore, these cell surface receptors are targets of the CAR of the presently claimed methods as further described herein.

The term “recombinant” means a cell or organism in which genetic recombination has occurred. It also includes a molecule (e.g., a vector, plasmid, nucleic acid, polypeptide, or a small RNA) that has been artificially or synthetically (i.e., non-naturally) altered by human intervention. The alteration may be performed on the molecule within, or removed from, its natural environment or state.

For example, a “recombinant protein” or “recombinant polypeptide” refers to a protein or a polypeptide having sequences that are not naturally joined together and/or that are not found joined together in nature. An amplified or assembled recombinant protein or polypeptide may be included in a suitable vector, and the resulting “recombinant vector” may be used to transform or transduce a suitable host or recipient cell of a subject. A recombinant polypeptide or protein may serve a coding or non-coding function (e.g., promoter, origin of replication, ribosome-binding site, etc.) as well. A “recombinant polypeptide” is one which is produced upon expression of a recombinant polynucleotide. Thus, the “recombinant protein,” the “recombinant polypeptide,” and the “recombinant vector” of the claimed methods are not products of nature or naturally occurring products.

The term “regulate” refers to either stimulating or inhibiting a function or activity of interest.

A “sample,” as used herein, refers preferably to a biological sample from a subject for which an assay or other use is needed, including, but not limited to, normal tissue samples, diseased tissue samples, sputum, mucus, phlegm, biopsies, cerebrospinal fluid, blood, serum, plasma, other blood components, gastric aspirates, throat swabs, pleural effusion, peritoneal fluid, follicular fluid, ascites, skin, hair, tissue, blood, plasma, cells, saliva, sweat, tears, semen, stools, Pap smears, and urine. A sample can also be any other source of material obtained from a subject who contains cells, tissues, or fluid of interest. A sample can also be obtained from cell or tissue culture.

As used herein, the term “secondary antibody” refers to an antibody that binds to the constant region of another antibody (the primary antibody).

As used herein, the term “solid support” relates to a solvent insoluble substrate that is capable of forming linkages (preferably covalent bonds) with various compounds. The support may be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it may be a known standard agent or compound which is administered and used for comparing results when administering a test compound, or it may be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a mouse or human.

As used herein, a “subject in need thereof” is a patient, animal, mammal, or human, who will benefit from the method of this invention. The mammal may be an animal. The mammal may also be a human or a mouse. The subject or patient may be female, such as a female subject or a female patient. The patient may also be a “pre-symptomatic patient,” meaning that the subject or patient has not yet experienced symptoms acknowledged to be associated with autoimmune disease.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity may be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

The phrase “suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions that will not affect the final peptide product.

The term “symptom,” as used herein, refers to any morbid phenomenon or departure from the normal in structure, function, observation, manifestation (e.g., clinical manifestation), or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

The term to “treat,” as used herein, means reducing the frequency with which symptoms are experienced by a patient or subject or administering an agent or compound to reduce the frequency with which symptoms are experienced.

As used herein, the term “transformation” encompasses all techniques in which a nucleic acid molecule may be introduced into a cell. Examples include, but are not limited to: transfection or transduction with viral vectors; transformation with plasmid vectors; electroporation; lipofection; microinjection (Mueller et al. (1978) Cell 15:579-85); Agrobacterium-mediated transfer; direct DNA uptake; WHISKERS™-mediated transformation; and microprojectile bombardment. These techniques may be used for both stable transformation and transient transformation of a host cell, such as a human or animal cell. “Stable transformation” refers to the introduction of a nucleic acid fragment into a genome of a host organism resulting in genetically stable inheritance. Once stably transformed, the nucleic acid fragment is stably integrated in the genome of the host organism and any subsequent generation. Host organisms containing the transformed nucleic acid fragments are referred to as “transgenic” organisms. “Transient transformation” refers to the introduction of a nucleic acid fragment into the nucleus or DNA-containing organelle of a host organism, resulting in gene expression without genetically stable inheritance.

As used herein, the terms “transduce” or “transduction” refer to a process where a virus transfers nucleic acid into a host cell, such as an animal cell (e.g., a murine or human cell).

As used herein, the term “transgene” refers to an exogenous nucleic acid sequence. In one example, a transgene is a gene sequence (e.g., an herbicide-resistance gene), a gene encoding an industrially or pharmaceutically useful compound, or a gene encoding a desirable agricultural trait. In yet another example, a transgene is an antisense nucleic acid sequence, wherein expression of the antisense nucleic acid sequence inhibits expression of a target nucleic acid sequence. A transgene may contain regulatory sequences operably linked to the transgene (e.g., a promoter, intron, 5′-UTR, or 3′-UTR). In some embodiments, a nucleic acid of interest is a transgene. However, in other embodiments, a nucleic acid of interest is an endogenous nucleic acid, wherein additional genomic copies of the endogenous nucleic acid are desired, or a nucleic acid that is in the antisense orientation with respect to the sequence of a target nucleic acid in a host organism.

The phrase “undesirable degradation,” as used herein encompasses any type of enzymatic, chemical or biochemical breakdown of the compound at its termini which is likely to affect the function of the compound, such as sequential degradation of the compound at a terminal end thereof.

A “variant”, as described herein, refers to a segment of DNA that differs from the reference DNA. A “marker” or a “polymorphic marker”, as defined herein, is a variant. Alleles that differ from the reference are referred to as “variant” alleles.

A “vector” is a composition of matter which comprises an isolated nucleic acid and which may be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

As used herein, the term “vector” refers to a nucleic acid molecule as introduced into a cell, thereby producing a transformed cell. A vector may include nucleic acid sequences that permit it to replicate in the host cell, such as an origin of replication. Examples include, but are not limited to, a plasmid, cosmid, bacteriophage, bacterial artificial chromosome (BAC), or virus that carries exogenous DNA into a cell. A vector may also include one or more genes, antisense molecules, selectable marker genes, and other genetic elements known in the art. A vector may transduce, transform, or infect a cell, thereby causing the cell to express the nucleic acid molecules and/or proteins encoded by the vector. A vector may optionally include materials to aid in achieving entry of the nucleic acid molecule into the cell (e.g., a liposome).

As used herein, the terms “cassette,” “expression cassette,” and “gene expression cassette” refer to a segment of DNA that may be inserted into a nucleic acid or polynucleotide at specific restriction sites or by homologous recombination. A segment of DNA comprises a polynucleotide containing a gene of interest that encodes a small RNA or a polypeptide of interest, and the cassette and restriction sites are designed to ensure insertion of the cassette in the proper reading frame for transcription and translation. In an embodiment, an expression cassette may include a polynucleotide that encodes a small RNA or a polypeptide of interest and may have elements in addition to the polynucleotide that facilitate transformation of a particular host cell. In an embodiment, a gene expression cassette may also include elements that allow for enhanced expression of a small RNA or a polynucleotide encoding a polypeptide of interest in a host cell. These elements may include, but are not limited to: a promoter, a minimal promoter, an enhancer, a response element, an intron, a 5′-UTR, a 3′-UTR, a terminator sequence, a polyadenylation sequence, and the like.

An “expression vector” refers to a vector comprising a recombinant polynucleotide comprising expression control sequences operatively linked to a nucleotide sequence to be expressed. An expression vector comprises sufficient cis-acting elements for expression; other elements for expression may be supplied by the host cell or in an in vitro expression system. Expression vectors include all those known in the art, such as cosmids, plasmids (e.g., naked or contained in liposomes) and viruses that incorporate the recombinant polynucleotide.

As used herein, the term “heterologous coding sequence” is used to indicate any polynucleotide that codes for, or ultimately codes for, a peptide or protein or its equivalent amino acid sequence, e.g., an enzyme, that is not normally present in the host organism and may be expressed in the host cell under proper conditions. As such, “heterologous coding sequences” may include one or additional copies of coding sequences that are not normally present in the host cell, such that the cell is expressing additional copies of a coding sequence that are not normally present in the cells. The heterologous coding sequences may be RNA or any type thereof (e.g., mRNA), DNA or any type thereof (e.g., cDNA), or a hybrid of RNA/DNA. Examples of coding sequences include, but are not limited to, full-length transcription units that comprise such features as the coding sequence, introns, promoter regions, 5′-UTR, 3′-UTR, and enhancer regions.

“Heterologous coding sequences” also include the coding portion of the peptide or enzyme (i.e., the cDNA or mRNA sequence), the coding portion of the full-length transcriptional unit (i.e., the gene comprising introns and exons), “codon optimized” sequences, truncated sequences or other forms of altered sequences that code for the enzyme or code for its equivalent amino acid sequence, provided that the equivalent amino acid sequence produces a functional protein. Such equivalent amino acid sequences may have a deletion of one or more amino acids, with the deletion being N-terminal, C-terminal, or internal. Truncated forms are envisioned as long as they have the catalytic capability indicated herein.

As used herein, the term “control” refers to a sample used in an analytical procedure for comparison purposes. A control can be “positive” or “negative”. For example, where the purpose of an analytical procedure is to detect a differentially expressed transcript or polypeptide in cells or tissue, it is generally preferable to include a positive control, such as a sample from a known animal or plant species exhibiting the desired expression, and a negative control, such as a sample from a known animal or plant species lacking the desired expression.

As used herein, the term “selectable marker gene” refers to a gene that is optionally used in transformation or transduction to, for example, protect plant cells from a selective agent or provide resistance/tolerance to a selective agent. In addition, “selectable marker gene” is meant to encompass reporter genes. Only those cells or plants that receive a functional selectable marker are capable of dividing or growing under conditions having a selective agent.

As used herein, the term “detectable marker” refers to a label capable of detection, such as, for example, a radioisotope, fluorescent compound, bioluminescent compound, a chemiluminescent compound, metal chelator, or enzyme. Examples of detectable markers include, but are not limited to, the following: fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In an embodiment, a detectable marker may be attached by spacer arms of various lengths to reduce potential steric hindrance.

As used herein, the term “detecting” is used in the broadest sense to include both qualitative and quantitative measurements of a specific molecule, for example, measurements of a specific polypeptide.

Unless otherwise specifically explained, all technical and scientific terms used herein have the same meaning as commonly understood by those of ordinary skill in the art that this disclosure belongs. Definitions of common terms in molecular biology maybe found in, for example: Lewin, Genes V, Oxford University Press, 1994; Kendrew et al. (eds.), The Encyclopedia of Molecular Biology, Blackwell Science Ltd., 1994; and Meyers (ed.), Molecular Biology and Biotechnology: A Comprehensive Desk Reference, VCH Publishers, Inc., 1995.

### Methods of the Present Disclosure

In autoimmune diseases, such as lupus, disease progression and severity correlates with the presence and/or increase of pathogenic autoantibodies in the subject. Since B cells produce antibodies, the inventors have identified that depletion of B cells in vivo and in vitro mitigates autoimmune diseases, such as lupus. The present methods utilize genetic technologies and methodologies to manipulate constructs, compositions, and components in order to effect positive change for subjects that have an autoimmune disease. The present methods generate cytotoxic T cell lymphocytes (CTLs), which are directed to recognize, attack, and/or kill pathogenic, mutant, and/or toxic antibody-producing cells (e.g. B lymphocytes) in a subject, particularly the cells responsible for the pathogenic autoimmune process in lupus disease manifestation. More specifically, the methods and techniques of the present disclosure engineer or “instruct” patient-derived cytotoxic T cells (CTLs) by inserting a treatment construct into the cytotoxic T cells, which bind and destroy B cells when the CTLs are reinfused or reinjected into the patient.

Importantly, the cytotoxic T cells (CTLs) of the present methods are engineered to express a chimeric antigen receptor (CAR) directed against B cells (anti-CD19, anti-CD20, and anti-BCMA) or plasma cells (anti-CD138). The CTLs are infected in vitro with retrovirus vectors encoding one of a number of embodiments of different CARs, and these cytotoxic T cells are subsequently infused into a recipient subject, such as a mouse or a human.

An illustrative recipient mouse is monitored to determine the development of such the lupus-like disease symptoms or clinical manifestations of the disease. Efficacy of the present therapeutic methods is successful in preventing, delaying, and/or reversing lupus manifestations in the subject or patient. Therefore, the claimed methods have direct application to autoimmune disease in human patients for treatment of diseases, such as Systemic Lupus Erythematosus (SLE), rheumatoid arthritis, diabetes (type I), scleroderma, Grave's Disease, and others autoimmune diseases that currently lack effective and focused therapies.

Thus, the present disclosure relates to methods of treating an autoimmune disease in a subject. The method of treating autoimmune diseases comprises, consists essentially of, or consists of administering one or more or a plurality of engineered cytotoxic T cells to the subject, wherein each of the plurality of engineered cytotoxic T cells comprises a recombinant vector that expresses a chimeric antigen receptor (CAR). The method further comprises depleting the number of antibody-producing cells, such as B cells, in the subject, and improving one or more clinical manifestations of the autoimmune disease in the subject.

The present methods utilize innovative genetic technology for ablating B cells. The technology uses engineered cytotoxic T cells expressing chimeric antigen receptors (CAR) to eradicate CD19 B cells from autoimmune subjects or patients. The present immunotherapeutic methodologies have been successfully developed to eliminate B cells responsible for autoimmune diseases, for example lupus.

Another embodiment of the present methods is related to a method of treating one or more clinical manifestations of lupus in a subject. The method comprises administering one or more or a plurality of engineered cytotoxic T cells to the subject, wherein each of the plurality of engineered cytotoxic T cells comprises a recombinant vector that expresses a chimeric antigen receptor. The method further comprises reducing or depleting the number of antibody-producing cells in the subject, wherein the ablation of B cells prevents, delays, or reverses one or more clinical manifestations of lupus in the subject. Therefore, this method treats one or more clinical manifestations of lupus in the subject, by preventing, delaying, or reversing one or more clinical manifestations of lupus in a subject. Separately, the present disclosure is directed to a method of preventing, delaying, or reversing one or more clinical manifestations of an autoimmune disease, such as lupus, in a subject.

In a further embodiment, a method of the present disclosure is related to reducing and/or depleting the number of antibody-producing cells in a subject. This method comprises administering one or more or a plurality of engineered cytotoxic T cells to the subject. Each one of the plurality of engineered cytotoxic T cells comprises a recombinant vector that expresses a chimeric antigen receptor. Subsequent to reinfusion of the engineered cytotoxic T cells into the subject, the CAR of the CTLs binds to antibody-producing cells (e.g., B cells) in the host, and kills those cells, thereby reducing or depleting the number of antibody-producing cells in the subject, and ultimately, improving disease manifestations or symptoms in the subject.

Another embodiment of a method of the present disclosure is related to a method of monitoring efficacy of lupus treatment in a subject. The method comprises measuring one or more clinical manifestations of lupus in the cells and/or tissues of a subject prior to treatment administration. The method further comprises administering a treatment construct comprising one or more or a plurality of engineered cytotoxic T cells to the subject. Each one of the plurality of engineered cytotoxic T cells comprises a recombinant vector that expresses a chimeric antigen receptor. At least one hour after treatment administration, the method comprises remeasuring the one or more clinical manifestations of lupus in the subject. Additionally, the method further comprises assessing the one or more clinical manifestations of lupus by determining the difference between the cells and/or tissues of the subject prior to treatment administration compared to the cells and/or tissues of the subject after treatment administration.

Another method encompassed by the present disclosure is one or more methods of diagnosing, prognosing, and/or monitoring the progression of an autoimmune disease in a subject or a patient. The method comprises assessing, measuring, and/or quantitating the symptoms and or clinical manifestations, including secondary effects, of the autoimmune disease in the subject, if applicable. Notably, the subject or patient may be pre-symptomatic, such that there are no symptoms and/or clinical manifestations of disease to initially assess. One or more clinical manifestations or “symptoms” of the autoimmune disease(s) treated by the present methods as experienced by the subject comprise, consist essentially of, or consist of proteinuria, alopecia, organ enlargement, hypercellular glomeruli, IgG tissue deposits, an increase in the quantity (i.e., number) or concentration of B cells, skin lesions, scabs and/or combinations thereof. Administering a treatment construct comprising one or more or a plurality of engineered cytotoxic T cells to the subject. Reassessing, remeasuring, and/or requantitating the symptoms and or clinical manifestations, including secondary effects, of the autoimmune disease in the subject, if applicable, after treatment. Based on a comparison and evaluation of the pre- and post-treatment disease manifestations, determining whether or not the subject has an autoimmune disease (i.e., diagnosing disease), determining whether the health of the subject is improving or worsening (i.e., prognosing disease), and/or ascertaining progression of the disease in the subject.

In addition, a method of the present disclosure relates to producing and/or engineering cytotoxic T cells for treatment of an autoimmune disease in a subject. This method comprises, consists essentially of, and/or consists of obtaining or extracting a T cell from a subject, wherein the T cell is competent to host exogenous, heterologous, and/or foreign nucleic acids, such as DNA, RNA or proteins that do not originate from the host or the host cell or tissue. The method also comprising transforming, transducing, or transfecting the T cell derived from subject with a vector, such as a recombinant vector. The recombinant vector of the present method comprises, consists essentially of, and/or consists of a chimeric antigen receptor (CAR) that binds to antibody-producing cells of the subject, such as B cells.

Additional diagnostic and/or mechanistic methods are also described in the present disclosure. For example, one embodiment off the present methods is directed to a method to detect and correct present and/or potential defects in the treatment of autoimmune disease in a subject. The method comprises utilizing, testing, experimenting, dosing, and/or investigating a mouse model of autoimmune disease (e.g., lupus), such as NZBxNZW F1 mice and/or MRL/lpr mice, to identify, detect, and/or correct any problems in treating humans for autoimmune disease. These mouse models for autoimmune disease may be utilized to detect and/or identify spontaneously mutated CD19 that may escape the targeting of anti-CD19 CAR T cells causing reduced efficacy in anti-CD19 CAR T-treated patients. Additionally, this method of utilizing mouse models for autoimmune disease would also enable identification of molecular, genetic, and/or selectable markers to assess the efficacy of CAR therapy in humans.

In addition, a virus delivery method or a method of delivering a virus to a subject, is also described by the present disclosure. This method comprises transducing the cytotoxic T cells with a recombinant vector that is a viral vector. The viral vector of the viral delivery method comprises the chimeric antigen receptor (CAR) that binds to antibody-producing cells of the subject, such as B cells, once the CTLs are reinfused into the subject.

Generally, the construct of the claimed methods may be an experimental and/or clinical therapeutic construct, such as a treatment construct. The treatment construct of the claimed methods is used for treating and/or relieving one or more symptoms and/or one or more clinical manifestation of a disease, such as an autoimmune disease, in a subject.

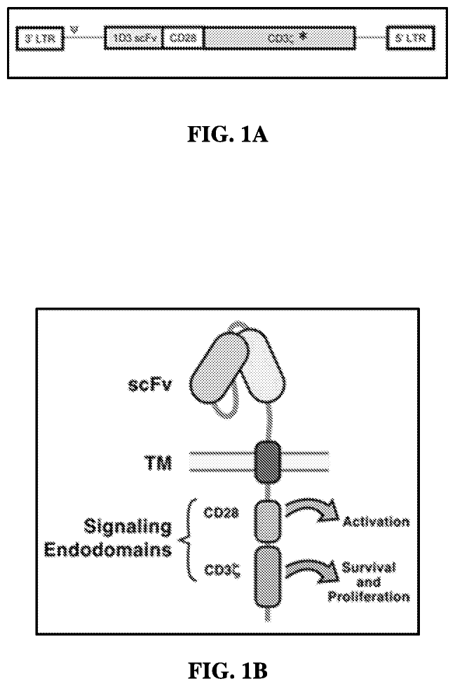

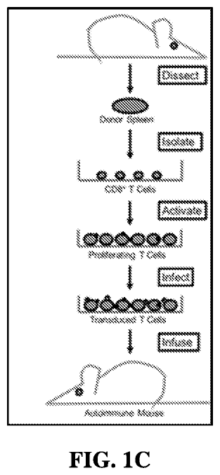

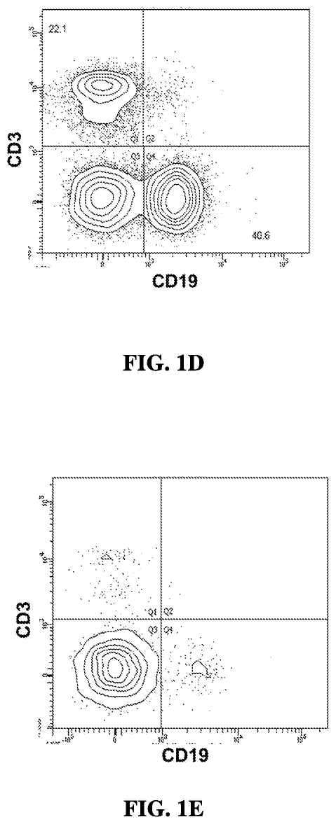

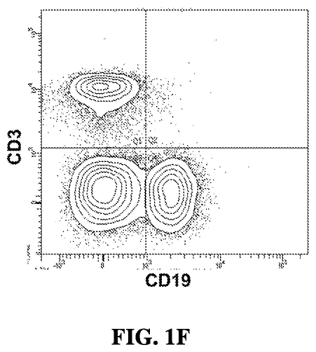

The construct of the claimed methods comprises, consists essentially of, or consists of a vector, such as a recombinant vector. The recombinant vector of the construct comprises, consists essentially of, or consists of one or more additional components, notably a chimeric antigen receptor. The chimeric antigen receptor (CAR) comprises, consists essentially of, or consists of an extracellular antibody binding domain, called the single-chain variable fragment (i.e., scFᵥ), along with additional intracellular signaling domains (FIG. 1A). Collectively, expression of the extracellular and intracellular domains of the chimeric antigen receptor (CAR) is primarily responsible for driving the cytotoxic activation, differentiation, and function (e.g., the regulation) of the recipient CTLs.

The construct comprising the CAR expression vector may be delivered to the cytotoxic T cells (CTLs) by any methods known in the arts. Through diligent design and testing, the expression vectors, transduction approaches, ex vivo T cell purification, and short term cell culture of the present methods were developed, refined, and/or optimized (“engineered”) for reinfusion of the engineered CTLs into subjects or patients and were determined to be safe and effective. For example, polymerase chain reaction (PCR), nucleic acid sequencing, transformation, and/or transduction are molecular techniques that are comprised within the claimed methods. In an illustrative embodiment, the CAR expression vector may be delivered to the CTLs by viral transduction in vitro. More specifically, the present methods comprise engineering via transduction of syngeneic T cells with retrovirus or lentivirus containing the chimeric antigen receptor (CAR) to produce cytotoxic T cells (CTLs).

The engineered cytotoxic T Cells (CTLs) comprising the CAR are reinfused back into the body of the subject, also called the recipient. In the body of the subject or the recipient, the engineered CTLs express the CAR, which recognizes a surface molecule on a target cell and induce its cell death, such as via apoptosis. An illustrative embodiment of the surface molecule of a therapeutic target of the chimeric antigen receptor (CAR) is CD19, a marker for B cells (i.e., the target cell). Additional exemplary embodiments of surface molecules on B cells that are targeted by the CAR of the present methods are CD20, CD138, and B-cell maturation antigen (BCMA). Because the CTLs differentiate into effector cells and/or plasma cells, they can perpetuate their cytotoxic function on therapeutic target cells in the body of the subject (e.g., B cells or plasma cells) many times over. Thus, the instant methods provide evidence that the utilization of engineered cytotoxic T cells comprising a chimeric antigen receptor (CAR) targeting B cells is an effective treatment for autoimmune diseases in a subject.

The autoimmune disease treated by the present methods comprises, consists essentially of, and/or consists of lupus, rheumatoid arthritis, diabetes, scleroderma, Grave's Disease, or combinations thereof. Lupus treated by the methods described herein may be Systemic Lupus Erythematosus (SLE) or other types of lupus (e.g., cutaneous lupus erythematosus, neonatal lupus, and/or drug-induced lupus erythematosus). Diabetes of the present method may be type I diabetes.

The disease or autoimmune disease treated by the present method is not a primary cancer. More specifically, types of primary cancer that are not treated by the present method include, but are not limited to melanoma, leukemia, lymphoma, breast cancer, prostate cancer, ovarian cancer, uterine cancer, cervical cancer, skin cancer, pancreatic cancer, colorectal cancer, renal cancer and lung cancer.

The subject of the present method is a patient, an animal, a mammal, such as a mouse or a human. The mammal may be an animal. The mammal or the animal may also be a human or a mouse or a plurality thereof (i.e., one or more humans and/or one or more mice). The subject or the patient may be female, such as a female subject or a female patient.

A mouse of the present method may be any murine animal, such as a mouse. In particular, the mouse of the present methods may be any mouse used as an experimental research or disease mouse model for autoimmune disease, such as for lupus or rheumatoid arthritis or any other autoimmune disease of interest. In an illustrative embodiment, the mouse of the present method may be a NZBxNZW mouse (e.g., a NZBxNZW F1 mouse and/or a NZBxNZW F1 female mouse) and/or a MRL/lpr mouse. However, the mouse of the present disclosure is not a C3H mouse or any other type of cancer mouse model.

One or more clinical manifestations or “symptoms” of the autoimmune disease(s) treated by the present methods as experienced by the subject comprise, consist essentially of, or consist of proteinuria, alopecia, organ enlargement, hypercellular glomeruli, IgG tissue deposits, an increase in the quantity (i.e., number) or concentration of B cells, skin lesions, scabs and/or combinations thereof. Proteinuria, as observed in subjects treated by the methods of the present disclosure, comprises an increase in the quantity or concentration of protein measured in the urine of the subject indicating improper kidney filtering function by the subject.

Alopecia is the abnormal and/or pathogenic loss of body hair of the subject. Subjects may also experience organ enlargement. For example, the subject may experience enlargement of the kidney or spleen due to accumulation of antibody-producing cells, such as B cells and T cells, in the diseased organs of the subject. In addition, the subject may experience hypercellular glomeruli and deposits of immunoglobulin (IgG) in the organ tissues (e.g., kidneys and spleen). An exemplary clinical manifestation of the autoimmune disease of the present methods comprises an increase in the quantity, concentration, and/or proliferation of the B cell population in the subject. In some embodiments of the instant methods, the B cells are plasma cells.

More specifically, the present disclosure is directed to methods comprising one or more or a plurality of cytotoxic T cells. Each one of the plurality of cytotoxic T cells of the claimed method is engineered to comprise a recombinant vector construct. The recombinant vector construct of the claimed method comprises, and therefore expresses one or more chimeric antigen receptor (CAR) to reduce and/or deplete the number of antibody-producing cells, such as B cells, in a subject in order to treat autoimmune diseases, such as lupus.

In the methods of the present disclosure, a plurality of cytotoxic T cells derived from a subject or patient have been engineered to express surface receptors. As previously described, the subject of the present methods may be a mammal or an animal, such as a mouse or a human. Accordingly, the one or more or the plurality of engineered cytotoxic T cells are murine cytotoxic T cells. Alternatively, the one or more or the plurality of engineered cytotoxic T cells are human cytotoxic T cells.

Exemplary surface receptors of the engineered cytotoxic T cells comprise one or more chimeric antigen receptors, which bind to antibodies. Illustrative antibodies that bind to the CAR domain of the engineered cytotoxic T cells (“CAR T cells”) include, but are not limited to, anti-CD19, anti-CD20, anti-CD138, and anti-BCMA (i.e., B-cell maturation antigen). In exemplary embodiments of the present methods, the cytotoxic T cells (“CAR T cells”) of the present methods are engineered to express a chimeric antigen receptor (CAR) directed against B cells (i.e., anti-CD19, anti-CD20, or anti-BCMA) or plasma cells (i.e., anti-CD138). The anti-CD19, anti-CD20, anti-CD138, and anti-BCMA CARs may be expressed in an inducible manner to assess the potential to transiently deplete B cells or plasma cells, respectively.

Upon binding of the antibody, anti-CD19 CAR receptors, for example, stimulate the release of cytotoxic mediators in the cytotoxic T cells (CTLs) toward the bound B cell. Autologous T cells comprising the chimeric antigen receptors (CARs) have shown remarkable efficacy in a growing number of B cell malignancies because they retain potency over time that can lead to lasting remission of diseases, such as cancer. More specifically, the reduction and/or depletion of antibody-producing cells, such as B cells, after reinfusion of CAR T cells in the body of a subject may be maintained long-term.

More specifically, depletion of B cells in a subject is stable and continuous for a time period, including but not limited, to at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 48 months, indefinitely, and for the remainder of the lifetime of the mouse. In addition, the depletion of B cells in the subject after administration of the treatment construct of the present methods is stable and continuous for a time period over 1 month, over 2 months, over 3 months, over 4 months, over 5 months, over 6 months, over 7 months, over 8 months, over 9 months, over 10 months, over 11 months, over 12 months, over 18 months, over 24 months, over 48 months, indefinitely, and for the remainder of the lifetime of the mouse. In particular, evidence of the present disclosure shows that B cells reduction and/or depletion in the subject treated by the present methods may be maintained indefinitely, for at least 3 months, for over 6 months, for at least 7 months, and for at least 12 months.

As previously described, the cytotoxic T cells of the present methods comprise a vector, such as a recombinant vector. In addition, the recombinant vector of the present methods further comprises, consists essentially of, or consists of a promoter and a gene, such as a transgene. The promoter of the recombinant vector is responsible for regulating and/or driving expression of the operably linked gene or transgene.

An exemplary embodiment of the recombinant vector construct of the claimed methods comprises, consists essentially of, or consists of SEQ ID NO: 1 and/or SEQ ID NO: 4. In addition, an embodiment of the recombinant vector of the present disclosure may comprise, consist essentially of, or consist of a polynucleotide having a sequence identity of at least 85%, 87.5%, 90%, 92.5%, 95%, 97.5%, 99%, 99.1%, 99.2%, 99.3%, 99.4%, 99.5%, 99.6%, 99.7%, 99.8%, 99.9%, and 100% sequence identity to SEQ ID NO: 1.

In another embodiment, the scFᵥ portion of the recombinant vector construct denoted by polynucleotide SEQ ID NO: 1 may encode a polypeptide that comprises, consists essentially of, or consists of SEQ ID NO: 4. In addition, an embodiment of the recombinant vector of the present disclosure may comprise, consist essentially of, or consist of a polypeptide having a sequence identity of at least 85%, 87.5%, 90%, 92.5%, 95%, 97.5%, 99%, 99.1%, 99.2%, 99.3%, 99.4%, 99.5%, 99.6%, 99.7%, 99.8%, 99.9%, and 100% sequence identity to SEQ ID NO: 4.

Illustrative embodiments of promoters of the recombinant vector of the present methods may comprise promoter elements from any gene and/or organisms known to regulate expression. For example, promoters of the claimed methods include, but are not limited to, promoters derived from the SV40, TRE3GS, PGK, and EF-1α genes. In addition, the promoters may be derived from viral origins, such as promoters from the murine leukemia virus (MLV) or the mouse stem cell virus (MSCV). An exemplary gene of the present treatment construct comprises, consists essentially of, or consists of the mCherry gene.

Further, the recombinant vector of the present treatment construct may further comprise long terminal repeats (LTRs). Illustrative LTRs of the recombinant vector may comprise, consist essentially of, or consist of 5′ LTRs and/or 3′ LTRs. An exemplary embodiment of the recombinant vector of the claimed methods may comprise both 3′ LTRs and 5′ LTRs.

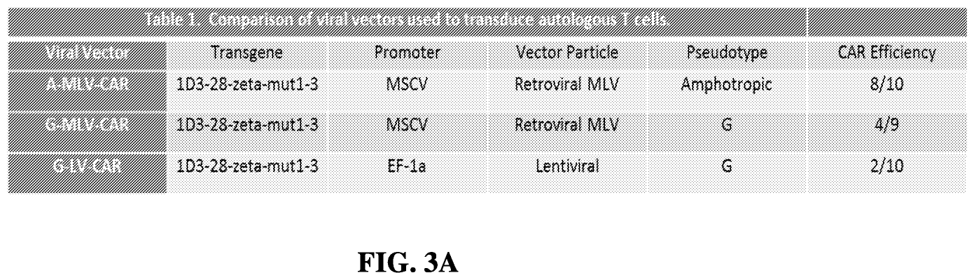

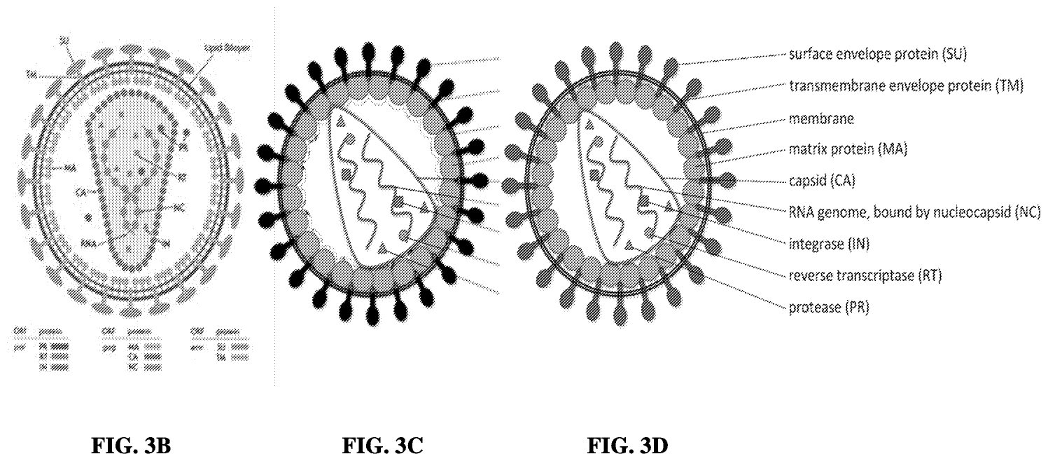

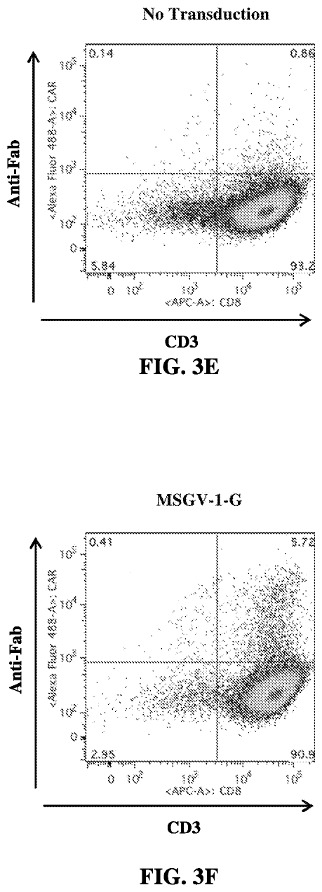

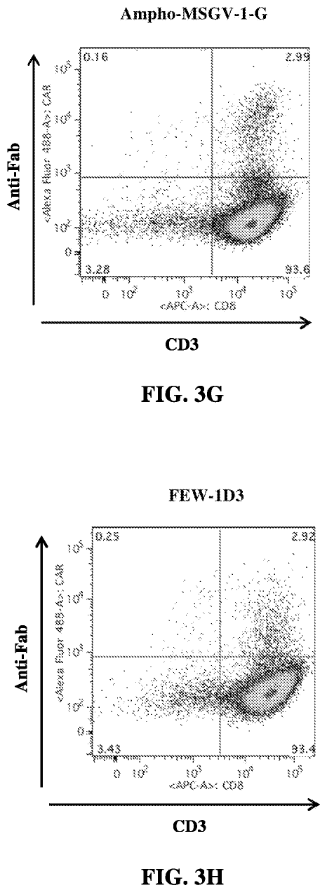

In one embodiment of the recombinant vector of the present method, the vector is a viral vector. Illustrative embodiments of a viral vector of the present methods comprise, consist essentially of, or consist of A-MLV-CAR, G-MLV-CAR, and/or G-LV-CAR (see FIG. 3A). The recombinant vector of the present method may also comprise a vector particle. For example, the vector particle of the present method may be a retrovirus or a lentivirus. Further, the pseudotype of the vector particle of the present methods may be an amphotropic envelope protein. Alternatively, the pseudotype of the vector particle of the present methods is an envelope glycoprotein from vesicular stomatitis virus.

Surprisingly, the presently described methods require as few as a single administration of the treatment construct comprising the engineered cytotoxic T cells to maintain reduction and/or depletion of the target cell populations (e.g., B cells) in the subject indefinitely. More specifically, the diagnostic, mechanistic, and/or treatment methods described in the present disclosure have the distinct advantage of requiring as few as only a single administration of the CAR T cells to the subject in order to effectuate indefinite mitigation of B cell populations in the treated subject. Alternatively, prior art methods have consistently required multiple administrations of the immunotherapy to the subject, wherein the multiple administrations typically comprise a first administration and one or more subsequent administrations of the immunotherapy to the subject in order to maintain reduction and/or depletion of the target antibody-producing cell population, such as the B cell population.

The present methods also enable the cytotoxic T cells to migrate and effectuate cell death and/or apoptosis of target cells at different sites in the recipient or subject. Further, the present methods allow the targeted antibody-producing cells (e.g., B cells) to develop into both effector and memory cell populations, and efficiently effectuate treatment in different areas of the body of the subject. Importantly, treatment of the present methods provide efficacy to subjects before the autoimmune disease manifests, as well as after overt autoimmunity has been established in the subject (e.g., after disease has been present in the subject for a significant time). In addition, polyclonal cytotoxic T cells expressing the CAR of the present methods achieve superior efficacy as compared to monoclonal antibodies to the same cellular targets in patients. Moreover, current treatments for autoimmune diseases, such as lupus, are generalized immuno-suppression regimens that have serious side effects, which are avoided with the treatment methods described herein.

### Methods of Administering Treatment Constructs to Humans

The treatment construct of the present disclosure may be administered in an experimental or clinical environment to a human subject as a therapeutic composition. The therapeutic composition of the present disclosure may also include an adjuvant or a pharmaceutically acceptable carrier. In one aspect, cytotoxic T cells comprising a recombinant vector that comprises a chimeric antigen receptor (CAR) that binds to B cells, are included in the therapeutic composition. In one aspect, the immunogenic therapeutic construct further comprises isolated viral peptides. Various aspects and embodiments of methods of administering the treatment construct of the present disclosure are described in further detail below.

Another embodiment of the present disclosure is directed to methods of preparation and use (i.e., administration) of a pharmaceutical composition. The therapeutic or compound of the present disclosure comprises a construct useful for the treatment of diseases, disorders, or infections, such as an autoimmune disease (e.g., lupus) as disclosed herein. In the present pharmaceutical or therapeutic construct, an antibody present on the CAR may be the compound or active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these.

Pharmaceutically acceptable carriers include physiologically tolerable or acceptable diluents, excipients, solvents or adjuvants. The compositions are preferably sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical or therapeutic compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g. 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions may be prepared in conventional forms, either as liquid solutions or suspensions, solid forms suitable for solution or suspension in liquid prior to injection, or as emulsions. Pharmaceutical compositions according to the present invention may be prepared in a manner fully within the skill of the art.

The therapeutic composition of the invention may include pharmaceutically acceptable salts thereof, or pharmaceutical compositions comprising these compounds may be administered so that the compounds may have a physiological effect. For example, in one embodiment of the present disclosure, a protein or peptide of the invention, or a combination thereof, may be administered to a subject by a route selected from, including, but not limited to, intravenously, intrathecally, locally, intramuscularly, topically, orally, intra-arterially, etc. Administration may also occur enterally or parenterally; for example orally, rectally, intracisternally, intravaginally, intraperitoneally, locally (e.g., with powders, ointments or drops), or as a buccal or nasal spray or aerosol. Parenteral administration is preferred. Particularly preferred parenteral administration methods include intravascular administration (e.g. intravenous bolus injection, intravenous infusion, intra-arterial bolus injection, intra-arterial infusion and catheter instillation into the vasculature), peri- and intra-target tissue injection (e.g. peri-tumoral and intra-tumoral injection), subcutaneous injection or deposition including subcutaneous infusion (such as by osmotic pumps), intramuscular injection, and direct application to the target area, for example by a catheter or other placement device. Controlled- or sustained-release formulations of a pharmaceutical composition of the invention may be made using conventional technology

The pharmaceutical compositions useful for practicing the invention may be administered to deliver a dose of between 1 ng/kg/day and 100 mg/kg/day.

Where the administration of the peptide is by injection or direct application, the injection or direct application may be in a single dose or in multiple doses. A pharmaceutical composition of the invention may also be prepared, packaged, and/or sold in bulk, such as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

However, where the administration of the compound is by infusion, the infusion may be a single sustained dose over a prolonged period of time or multiple infusions. Notably, an exemplary embodiment of the methods of the present disclosure comprise as few as only a single administration of the treatment construct or therapeutic composition to a subject or patient without the need for multiple administrations or infusions for the subject to achieve and maintain efficacy of disease treatment (e.g., autoimmune diseases, such as lupus).

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

In addition to ex vivo administration of the present compositions, the present disclosure also describes in vivo methods of treating a subject. The methods described herein comprise, consist of, and consist essentially of administering a pharmaceutical or therapeutic composition of the present disclosure comprising at least one compound of the present invention to a subject. In particular, the methods of the present disclosure are directed to administering the protein, peptide fragments, and/or antibody described herein to a subject for treatment of a disease, disorder, or an infection. More specifically, the compositions and methods of the present disclosure are directed to a method of treating autoimmune diseases, such as lupus, by administering the constructs and compositions of the present disclosure to a subject. Compounds identified by the methods of the invention may be administered with known compounds or in combination with other medications as well. In accordance with one embodiment a method of treating autoimmune disease, such as lupus in a subject or a patient is provided wherein the method comprising administering cytotoxic T cells comprising a chimeric antigen receptor (CAR) that binds to B cells, as disclosed herein to the patient.

It will be understood by the skilled artisan that such pharmaceutical compositions are generally suitable for administration to animals of all sorts. Subjects to which administration of the pharmaceutical compositions of the invention is contemplated include, but are not limited to, humans and other primates, mammals including commercially relevant mammals, such as mice, cattle, pigs, horses, sheep, cats, and dogs, birds, including commercially relevant birds such as chickens, ducks, geese, and turkeys.

Typically, dosages of the compound of the invention which may be administered to an animal, preferably a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. In one aspect, the dosage of the compound will vary from about 1 mg to about 10 g per kilogram of body weight of the animal. In another aspect, the dosage will vary from about 10 mg to about 1 g per kilogram of body weight of the animal.

One major benefit of the methods of the present disclosure is single administration of the treatment construct or therapeutic composition of the present method comprising cytotoxic T cells comprising a chimeric antigen receptor (CAR) that binds to B cells, as disclosed herein, to the patient or subject to achieve and/or maintain efficacy (i.e., reduction and/or prevention of clinical manifestations of autoimmune diseases). However, if necessary, the compound may be administered to a subject (e.g., an animal or human) as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc. However,

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Suitable preparations of the pharmaceutical compositions described herein include injectables, either as liquid solutions or suspensions, however, solid forms suitable for solution in, suspension in, liquid prior to injection, may also be prepared. The preparation may also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

In addition to the active ingredient, a pharmaceutical composition of the invention may further comprise one or more additional pharmaceutically active or inactive components or agents. Particularly contemplated additional agents include anti-emetics and scavengers, such as cyanide and cyanate scavengers. Other additional ingredients may include, but are not limited to, one or more of the following: excipients; surface active agents; dispersing agents; inert diluents; granulating and disintegrating agents; binding agents; lubricating agents; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other additional ingredients that may be included in the pharmaceutical compositions of the invention are known in the art and described, for example in Genaro, ed., 1985, Remington's Pharmaceutical Sciences, Mack Publishing Co., Easton, Pa.

In other embodiments, therapeutic agents and pharmaceutical compositions of the present disclosure, include, but not limited to, cytotoxic agents, anti-angiogenic agents, pro-apoptotic agents, antibiotics, hormones, hormone antagonists, chemokines, drugs, prodrugs, toxins, enzymes or other agents may be used as adjunct therapies when using the multimeric peptide ligand complexes described herein. Drugs useful in the invention may, for example, possess a pharmaceutical property selected from the group consisting of antimitotic, antikinase, alkylating, antimetabolite, antibiotic, alkaloid, anti-angiogenic, pro-apoptotic agents, and combinations thereof. Techniques for detecting and measuring these agents are provided in the art or described herein.

Other embodiments of the invention will be apparent to those skilled in the art based on the disclosure and embodiments of the invention described herein. It is intended that the specification and examples be considered as exemplary only, with a true scope and spirit of the invention being indicated by the following claims. While some representative experiments have been performed in test animals, similar results are expected in humans. The exact parameters to be used for injections in humans may be easily determined by a person skilled in the art. Other techniques known in the art may be used in the practice of the present invention.

The invention is now described with reference to the following Examples. Without further description, it is believed that one of ordinary skill in the art can, using the preceding description and the following illustrative examples, make and utilize the present invention and practice the claimed methods. The following working examples therefore, are provided for the purpose of illustration only and specifically point out the preferred embodiments of the present invention, and are not to be construed as limiting in any way the remainder of the disclosure. Therefore, the examples should be construed to encompass any and all variations which become evident as a result of the teaching provided herein.

### EXAMPLES

While this invention has been disclosed with reference to specific embodiments, it is apparent that other embodiments and variations of this invention may be devised by others skilled in the art without departing from the true spirit and scope of the invention. Examples of the present methods comprise cytotoxic T cells (CTLs) expressing a specific chimeric antigen receptor (CAR) in mouse models of autoimmune diseases, such as lupus. More specifically, transformation or transduction of CTLs in vitro with anti-CD19, anti-CD138, and anti-BCMA CAR and administration of the engineered CTLs to mice, such as NZBxNZW F1 mice since lupus manifestations in these mice are largely, if not entirely dependent on B cells.

Transduced murine CD8+ T cells comprising anti-CD19 CAR CTLs were infused into recipient NZBxNZW F1 mice at 6 weeks of age and at 8 months of age. Clinical disease manifestations and symptoms were observed, including IgM and IgG antibody titers and antinuclear antibody in serum, circulating B cell numbers, and proteinuria in animals up to 1 year of age. At the end of the experimental study, mice were sacrificed. Cells and tissues from mice organs, such as kidneys and spleens, were prepared for light microscopy and immunofluorescence analysis of immune complex deposition and hypercellularity. Depletion of B cells in the subject, depending on the stage of disease, prevented the initial development of disease symptoms, such as lupus, or arrested, prevented, and/or reversed further progression of disease or disease manifestations.

### Animals—Murine Disease Models

Animal welfare was monitored and euthanasia was induced in strict accordance with UTHSC Institutional Animal Care and Use committee approved protocols. 7-10 weeks old, female NZBxNZW F1 or MRL/lpr mice (Jackson Labs) were used in all studies. Two days prior to the injection of CAR-virus injected or control T cells, mice were irradiated with about 5-10 Gy, such as 5.0-5.5 Gy, including about 5.0, 5.1, 5.2, 5.3, 5.4, 5.4, 5.6, 5.7, 5.8, 5.9, 6.0, 7.0, 8.0, 9.0, and about 10.0 Gy to achieve transient myeloablation. For example, FIGS. 1D-1F are contour plots showing that mice blood lymphocyte levels recovered from transient myeloablation with 5.2 Gy of irradiation and had normal levels of CD19+ B cells as compared to CD3+ T cells within 18 days post-irradiation. These data demonstrate that the myeloablation of B cells did not permanently damage the mouse models prior to CAR treatment.

### Construction and Use of Retrovirus and Lentivirus Transfer Vectors

All recombinant DNA protocols were approved by the UTHSC Institutional Biosafety Committee. The anti-mouse CD19 single chain variable fragment (scFᵥ) was derived from the 1D3 lgG2a/κ rat hybridoma. The extracellular scFᵥ coding sequence was linked to a transmembrane domain of mouse CD28 and to two intracellular signaling domains. One intracellular domain was derived from CD28, and the other intracellular domain was an optimized version of the CD3ζ (zeta) signaling domain.

The coding sequence was inserted between two retroviral long-terminal repeats (LTRs) and placed downstream of a retroviral packaging signal to yield the MSGV-1D3-28Z.1-3 expression plasmid (Genbank Accession No. HM754222). All plasmid constructs were grown in recombination deficient E. coli (K12 derivative strains) by using standard molecular biology techniques known in the art. All vector DNAs used for transfection of mammalian cells were prepared from the transformed E. coli cultures using QIAGEN Plasmid Midi Kit.

The MSGV-1D3-28Z.1-3 retrovirus transfer plasmid was expressed in 293FT cells (Invitrogen). Retrovirus particle production was induced by co-transfection of the 1D3-28Z.1-3 plasmid with two additional plasmids that complement in trans the assembly of the virus. One of the plasmids (e.g., pCAGGS Gag-Pol) contributed the retroviral Gag-Pol fusion protein, while the other plasmid (e.g., pcLEco or pEco-Env) encodes the ecotropic murine virus surface glycoprotein.

To construct 1D3 CAR coding sequences in lentivirus, the anti-mouse CD19 sequences were inserted in the polylinker sequence of the pFEW lentiviral transfer plasmid (Addgene). The resulting transfer plasmid was expressed in 293FT cells (Invitrogen). Lentivirus particle production was induced by co-transfection with three additional plasmids that complement in trans the assembly of the lentivirus particles.

Two of the plasmids comprise the ViraPower system from Invitrogen. One of the plasmids (e.g., pLP1) contributes the lentiviral Gag-Pol fusion protein. The second plasmid (e.g., pLP2) contributes the lentiviral Rev protein. The third plasmid encodes the surface glycoprotein (e.g., ecotropic retrovirus gp70 protein or the SV-G glycoprotein).

Transfections of 293FT cells were carried out using Lipofectamine 2000 (Invitrogen). Virus was collected at 48-72 hours after transfection, and cell supernatants were filtered and concentrated to yield high-titer stocks, which, for the retrovirus, were tested for absence of replication competent virus.

Purification of CD8+ T Cells from Spleen

Spleen organs were harvested from age and strain-matched donor mice, minced, and dispersed into single cells in RPMI-10 (RPMI-1640 with 10% FCS) by pressing between sterile glass slides. Cell clumps and debris were removed by filtration through a 40 μm pore-size cell strainer. The cells were washed in RPMI-10 and red blood cells (RBCs) were lysed using the mouse erythrocyte lysis kit (R&D Systems, catalog WL2000).

CD8+ cells were enriched by negative selection on a MagCellect Mouse CD8+ T cell isolation kit (R&D Systems, Cat # MAGM203) following instructions from the manufacturer. The yield of CD8+ cells was approximately 10⁷ per spleen and 95% of the isolated cells expressed CD8a. The cells were cultured at 1 million per ml of RPMI-10 media with recombinant mouse IL-2 (R&D Systems, Cat #402-ML) at 30 I.U. per ml. CD28/CD3 beads (Gibco; Mouse T-activator anti-CD3/CD28 Dynabeads) were added at a 1:1 ratio with the CD8+ T cells.

### Transduction of CAR Transgenes

T cells were exposed to two consecutive virus infections. The first infection was carried out at 6 hours following plating of cells with activator beads. The second infection occurred at 24 hours.

Six-well tissue culture plates were coated with 2.5 ml/well of Retronectin (Clontech, catalog T100B) in sterile PBS, without Ca and Mg, and at a final concentration of 9 μg/ml. Following coating, plates were washed with sterile PBS and blocked with 2% BSA in PBS for 30 min. Following another wash, plates were incubated with 1.5 ml of RPMI-10 containing 40 μl of 200× concentrated virus stocks. The plates were centrifuged at 2000 rpm at 22-24° C. for 2h.

Following centrifugation, about 0.5 ml of the media was left in the well and 1 ml of the activated T cells, along with the activator beads was added. The second infection was performed exactly as the first, by using freshly prepared tissue culture plates. Cells were fed fresh media containing IL-2 every other day until they reached a 5-fold expansion from the initial cell numbers on the 5^{th} day of culture.

At that time, remaining activator beads were magnetically removed and discarded, and CD8⁺ T cells were washed, counted, and suspended at 1.2×10⁷ per ml in RPMI-1640 containing 10% autologous mouse serum just prior to injections into recipients. An aliquot of the cells was assayed for CAR expression by anti-rat Fab antibody, and 5-15% of cells were stained positive (no data shown). Approximately 100 μl of cell suspension was injected into the retroorbital sinus of each mouse with appropriate sedation and administration of analgesics. Immediately after the injection, the mice were administered 45,000 I.U. of IL-2 into the scruff of their neck.

### Flow Cytometry

Following injection of CD8+ T cells, mice were bled at periodic intervals from the retroorbital plexus, and heparinized blood was collected. Seventy microliters (μl) of blood was used to phenotype circulating lymphocytes using antibodies for CD3ζ, CD4, CD8a, and CD19. Before addition of antibodies, blood was incubated with 2 μl of mouse Fc block (Affymetrix, Catalog #14-0161) in 35 μl of HBSS to reduce nonspecific binding. After 10 min in block, the antibody cocktail was added, and the cells were incubated on ice for 30 min.

Following labeling, RBCs were lysed with 750 μl of high-yield lyse solution (Life technologies, Catalog # HYL250). After 10 mins of incubation, the cells were centrifuged at 1200 rpm for 10 min, re-suspended in 200 μl of the lyse solution, and acquired on BioRad ZE5 instrument. To assess the relative proportion of CD19-positive cells, forward and side scatter plots were used to define the lymphocyte gate, followed by further subdivision of lymphocyte subsets according to the specific purpose of each experiment.

CAR expression of engineered primary T cells was determined by staining with AF488-conjugated mouse anti-rat Fab fragment specific antibodies (Jackson ImmunoResearch Catalog #121-545-106). Ghost Dye Red710 (Tonbo Catalog #13-0871) was used to differentiate between live and dead cells.

### Serology and ELISA

Subject plasma was extracted and prepared by centrifugation for 10 minutes at 2,000 g and subsequently used to determine the levels of circulating total IgG, IgM, and anti DNA antibodies present. Total amount of immunoglobulins in the mouse plasma was determined by ELISA by coating with AffiniPure Goat Anti-Mouse IgG (H+L) (Jackson Immuno Research Lab-Catalogue #115-005-003). Antigen-coated plates were emptied and washed once with blocking buffer and then incubated for 1h at RT in the blocking buffer.

For mouse IgG detection, ten-fold serial dilutions of the subject plasma were prepared in blocking buffer (ranging from 10⁻² to 10⁻⁷), added to the plates, and incubated at RT for 3h. The plasma dilutions were aspirated and the plate was washed four times with PBS containing Tween20 (PBS-T). The detection antibody i.e., alkaline phosphatase labelled Goat antiMouse IgG at 1:1000 dilution in blocking buffer) was added and incubated for 1h at RT.

Secondary antibodies were aspirated and the plates were washed four times with PBS-T. Phosphatase detection substrate (Sigma-50942) at 1 mg/ml was added to the plate and incubated for color development over 20-30 min. Absorption in plates was read at 405 nm in an ELISA reader.

With each ELISA, a positive standard (mouse IgG, Catalog #015-000-0030) was used to control for interexperimental variations. Anti DNA IgG and IgM antibody titers were determined using ELISA plates that were coated with calf thymus DNA (Sigma, cat # D3664) at 10 μg per ml of PBS, supplemented with 1 mM EDTA. The same protocol was followed for isotype ELISAs except that 3-fold serial dilutions were prepared starting at a 1:30 initial dilution (range 1:30-1:7290).

RT PCR and q-PCR

To detect CAR T cells in circulation and in various tissues, total RNA was isolated from different mouse organs such as spleen, kidney, bone marrow, and whole blood using Trizol reagent (Invitrogen, catalog #15596018). Total RNA was prepared and freed of the contaminating DNA by DNase treatment and then quantified by a spectrophotometer. The total RNA was converted to cDNA using Transcriptor First strand cDNA kit (Roche-Catalog #04379012001) and qPCR was performed on a Lightcycler (Roche) using gene-specific primers and probes designed using Roche's Universal Probe Library Assay Design Center (https://qper.probefinder.com/organism.jsp).

The genes used for gene expression quantification were mouse TBP (as a housekeeping gene) and CD19CAR using primers spanning mouse genes, CD28 and the CD3ζ (zeta) junction (jxn). The forward primer to amplify the CD28-CD3ζ (zeta) junction (jxn) had the following primer sequence: GACTTCGCCGCCTACAGA (SEQ ID NO: 2). The reverse primer to amplify the CD28-CD3ζ (zeta) jxn had the following primer sequence: TCGTTGAACAGCTGGTTGG (SEQ ID NO: 3). The primers were coupled with Probe #21 from the Universal Probe Set (Roche) in order to detect amplified DNA that hybridized to the probe.

### Tissue Preparation and Pathology

Kidneys, spleen, ovaries, and other tissues were flash frozen in O.C.T. compound (Fisher Biotech, Cat #23-730-571) and sectioned at 5 μm thickness on a cryotome. Frozen sections were thawed and blocked in blocking buffer. The tissue sections were then reacted with goat anti-mouse IgG isotype antibodies for fluorescence microscopy.

Skin sections were formalin-fixed, paraffin-embedded, sectioned, and stained with Hematoxylin and eosin (H&E) stain, prior to microscopy. Skin pathology was evaluated, and proteinuria was determined by placing a 5 μl drop of freshly collected urine onto an Albustix urine analysis indicator strip. Comparison of the resulting color to a standard color scale provided by the manufacturer (Siemens, cat #2191) was utilized to determine proteinuria of the subject.

### Example 1: Expression of a Retrovirus and Expression Constructs

A diagram of the DNA construct for an exemplary chimeric antigen receptor (CAR) (e.g., anti-CD19-28Z.1-3 CAR called “anti-CD19 CAR”) that was incorporated into murine and human cytotoxic T cells (CTLs) of the present disclosure is shown in FIG. 1. FIG. 1A is a schematic of the structure of the retroviral expression cassette of the CAR. In particular, long terminal repeats (LTRs) flank the 5′ and 3′ ends of the construct, with “Ψ” indicating the retroviral packaging signal. The 1D3 anti-CD19 CAR construct comprises, consists essentially of, and consists of the 1D3 extracellular single-chain Fv domain upstream of the CD28 transmembrane and cytoplasmic signaling domains, followed by a variant CD3ζ (zeta) C-terminus (see FIG. 1A). The functional extracellular and intracellular membrane domains spanning the transmembrane domain (“TM”) of the anti-CD19 CAR fusion protein are also shown in FIG. 1B.

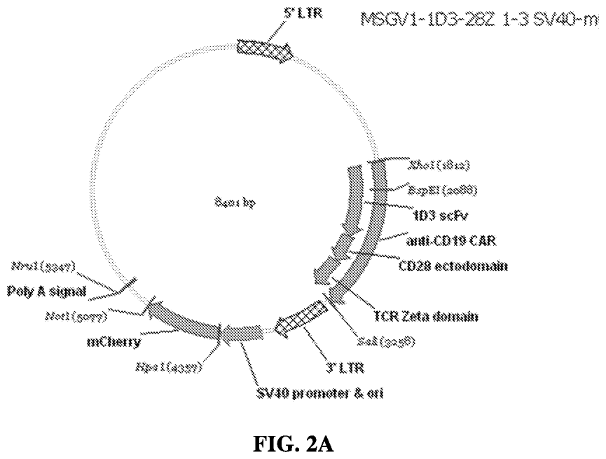

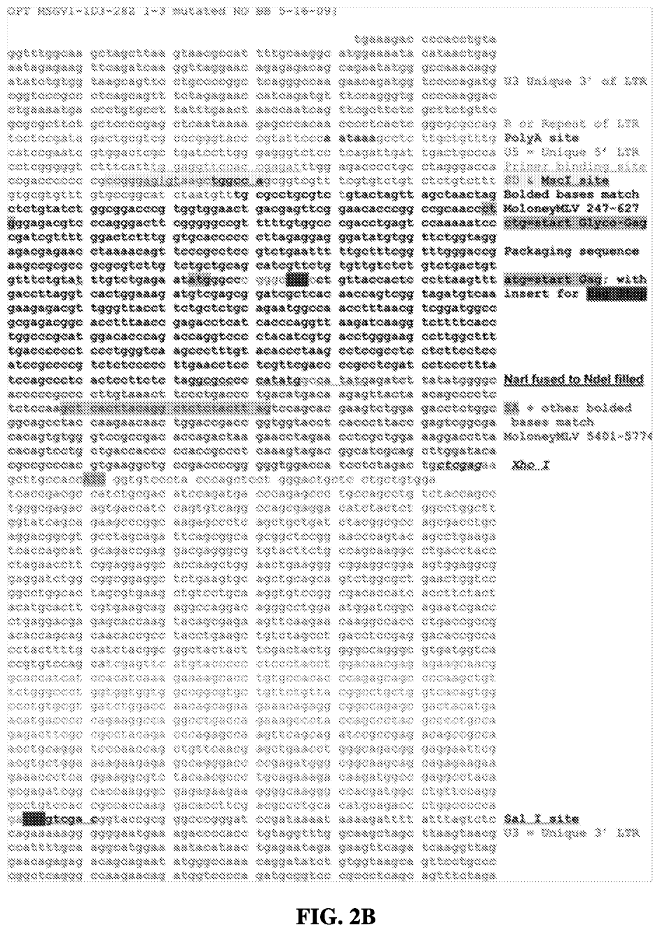

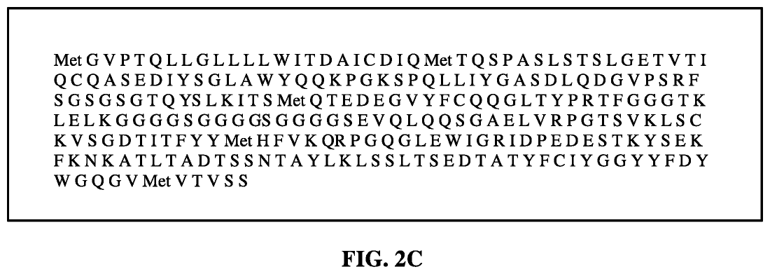

The single-chain variable fragment (scFᵥ) of the fusion protein that provides the CAR component of the present method constructs with its anti-mouse specificity was originally derived from the rat antibody, 1D3, IgG2a/κ hybridoma (see FIG. 1B). FIG. 2C (SEQ ID NO: 4) is a protein sequence showing an embodiment of the scFᵥ component of a CAR of the present methods.

The coding sequence links the extracellular scFᵥ to a transmembrane domain and to two additional intracellular signaling domains. The first intracellular domain is derived from CD28, the second intracellular domain is an optimized version of the CD3ζ (zeta) signaling domain. In this embodiment, conserved tyrosine residues in two of the three ITAMs from the CD3ζ (zeta) signaling domain were mutated to alanine residues, which reduce the activation of CAR-T cells, and thus decreases exhaustion leading to an increased persistence of the engineered T cells in vivo.

The coding sequence was inserted into the vector sequence between two retroviral long-terminal repeats (LTRs), a 5′ LTR and a 3′ LTR. In addition, the fusion protein further comprises a retroviral packaging signal to yield the recombinant 1D3-28Z.1-3 expression plasmid vector. An embodiment of the recombinant vector of the claimed methods has GenBank Accession No. HM754222.

An MSGV-1D3-28Z.1-3 embodiment of a retroviral vector of the present methods is expressed in 293FT cells by co-transfection with two additional plasmids that complement in trans the assembly of the virus. One of the additional plasmids contributes the Gag-Pol fusion protein, and the other encodes the pcLEco ecotropic murine virus surface glycoprotein. The transfections of 293FT cells using Lipofectamine 2000 (Invitrogen) were performed and virus was collected at 48-72 hours after transfection. Cell supernatants were purified and concentrated to yield a high-titer stock. This stock was tested for the absence of recombination competent virus before proceeding with additional experiments and evaluation.

Several embodiments of the recombinant vector were produced and tested in the present methods. As described in FIG. 1C, some method embodiments of the present disclosure comprise utilization of a retrovirus versus a lentivirus in the recombinant vector comprised within the engineered cytotoxic T cells of the claimed methods. Other embodiments include varying culture conditions of the cytotoxic T cells or differing injection procedures of the engineered cytotoxic T cells back into a subject or patient. For example, the several different types of mice subjects were used in the present methods, including NZBxNZW F1 and MRL/lpr mice, which are both models for autoimmune disease, such as lupus. As recipients, we used 7-month-old NZBxNZW F1 female mice, as female mice of that age overtly manifest autoimmune disease. We used anti-DNA titers to randomize mice to one of four experimental groups comprising about 10 mice each.

FIG. 2A provides a plasmid map of the functional domains in a recombinant vector embodiment of the present methods used to express anti-CD19 CAR. This specific embodiment of the MSGV1-1D3-28Z 1-3 SV20-m recombinant vector of the claimed methods comprises an SV40 promoter, a mCherry gene, both a 5′ and a 3′ LTR, along with the anti-CD19 scFᵥ. FIG. 2B shows the sequence of the regulatory and coding regions of the recombinant vector construct of FIG. 2A (SEQ ID NO: 1).

More specifically, the recombinant vector or recombinant plasmid of the present methods has functional portions derived from different organismic sources that are not found together in nature. For example, in some embodiments, the structural backbone of the plasmid is prokaryotic (e.g., bacterial), such as the origin of replication and the ampicillin resistance gene. Other portions of the plasmid embodiment are eukaryotic, such as the long terminal repeats (LTRs) and the mouse leukemia virus (MLV), which is a retrovirus that is endogenous to mouse (i.e., Mus musculus).

The chimeric antigen receptor (CAR) gene of the plasmid of the present disclosure comprises sequences from various different sources. For example, the single chain Fᵥ (scFᵥ) portion is derived from a rat antibody, called 1D3. FIG. 2C (SEQ ID NO: 4) shows a protein sequence for one embodiment of the scFᵥ of a CAR of the present methods. The plasmid embodiment further comprises portions of the mouse genes, CD28 and CD3ζ (zeta).

This CAR construct was expressed in three different virus delivery embodiments, including eco- and amphotropic retrovirus, as well as G-pseudotyped lentivirus for delivery to CTL (see FIGS. 3A-3D). As shown in Table 1, the virus delivery constructs A-MLV-CAR and G-MLV-CAR vector capsids are derived from murine leukemia virus (MLV), which only differ in the envelope protein that pseudotypes the vector particles: “A” denotes the amphotropic envelope protein and “G” denotes the envelope glycoprotein from vesicular stomatitis virus (VSV). The third vector embodiment, G-LV-CAR, transduces the same anti-CD19 CAR gene, but differs from the first two vector embodiments in that transcriptional control of the CAR is under the human EF-1α promoter, and the vector particles are lentivirus coated with the G envelope pseudotype (see Table 1 and FIG. 3A).

TABLE 1
Comparison of viral vectors used to transduce autologous T cells.

| | | | | | CAR |
|---|---|---|---|---|---|
| Viral Vector | Transgene | Promoter | Vector Particle | Pseudotype | Efficiency |
| A-MLV-CAR | 1D3-28-zeta-mut1-3 | MSCV | Retroviral MLV | Amphotropic | 8/10 |
| G-MLV-CAR | 1D3-28-zeta-mut1-3 | MSCV | Retroviral MLV | G | 4/9 |
| G-LV-CAR | 1D3-28-zeta-mut1-3 | EF-1α | Lentiviral | G | 2/10 |

For in vitro transduction, CD8+ selected T cell lymphocytes were infected with anti-CD19 CAR constructs comprising one of the three embodiments of the recombinant vectors. At the end of 72 hours post-infection, cell cultures were stained with anti-CD3 antibody (x-axis) and anti-Fab (y-axis) to identify T lymphocytes that expressed the extracellular portion of the anti-CD19 CAR. As seen in FIGS. 3F-3H, only about 3-6% of cells were identified using flow cytometry as being positive for anti-Fab, which identifies anti-CD19 CAR expression. Conversely, FIG. 3E shows that only a negligible amount (i.e., about 0.86%) of anti-Fab staining for B cells was observed in untransduced control cells of which over 93% stained for CD3+ T cells.

Therefore, NZBxNZW F1 mice cells comprising different embodiments of the recombinant vector of the claimed methods showed different transfection efficiencies. These results provided an opportunity to explore the cause for the differences in transfection efficiencies and the claimed method approach. At 11 weeks after CAR T cell administration, the three experimental groups showed substantial differences in the efficacy of B cell depletion, even though they had received the identical anti-CD19-28Z.1-3 gene. Because all three viral vectors transduced similar numbers of CD8+ T cells in vitro, and equal numbers of CAR CTLs were injected into recipient mice, the observed differences in B cell depletion in the mice must have derived from: (a) differences between the promoters driving the CAR expression, (b) the virus particle type, and/or (c) the envelope protein pseudotypes coating the vector.

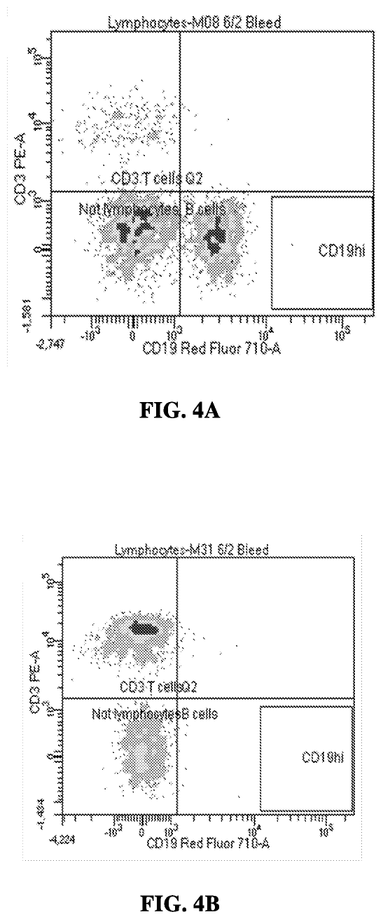

FIGS. 4A and 4B show detection of main lymphocyte populations in the blood of NZBxNZW F1 mice. FIG. 4A demonstrates that control NZBxNZW F1 mice had similar levels of CD19+ B cells and CD3+ T cells. However, following administration of anti-CD19 CAR CTLs to the NZBxNZW mice, the B cells were reduced and/or depleted to near background levels (see FIG. 4B). In mice that were successfully reconstituted with CAR-expressing CTL, B cell depletion was maintained for over 1 month, over 2 months, over 3 months, over 4 months, over 5 months, over 6 months, over 7 months, over 8 months, over 9 months, over 10 months, over 11 months, over 12 months, over 18 months, over 24 months, over 48 months, indefinitely, and for the remainder of the lifetime of the mouse. In illustrative embodiments, stable B cell depletion was maintained in CAR T cells for over 6 months, or for the remainder of their lifetime until mice were sacrificed for analysis.

### Example 2: In Vitro Expression of B Cells in C3H Cancer Mice

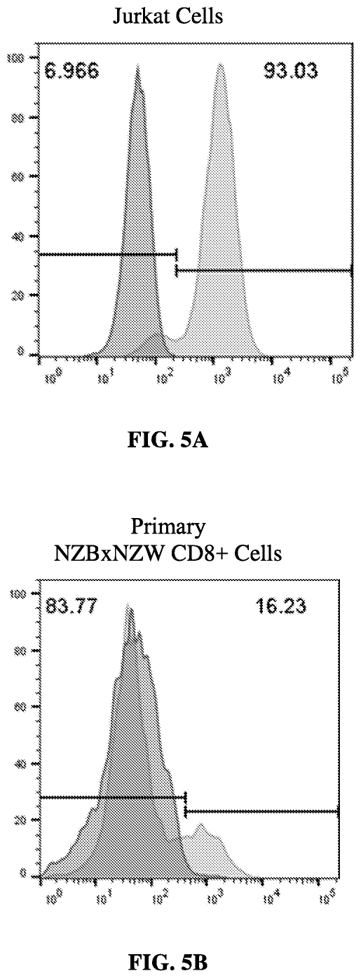

B cell depletion in NZBxNZW F1 mice affected with lupus, was expected to be as efficient as it was in C3H cancer mice, as shown in FIGS. 5A-5D. More specifically, a rapid decrease in B cell numbers and circulating Ig levels was observed in C3H mice that received anti-CD19 CAR CTLs as compared to control mice that did not receive the anti-CD19 CAR CTLs. Notably, the mouse of the present disclosure is not a C3H mouse or any other type of cancer mouse model.

However, less known, understood, or predictable is the outcome of administering engineered CTL to mice exhibiting autoimmune disease symptoms, such as lupus-like disease symptoms. Because the C3H cancer mice showed no adverse effects following CTL infusions, groups of 10 NZBxNZW F1 mice provided a sufficient number of animals for statistical assessment and analysis of autoimmune diseases. In prediseased and/or pre-symptomatic mice, administration of the engineered CTLs prevented development of clinical manifestations of autoimmune diseases, such as immune deposits in kidneys and thereby blocked or delayed glomerulonephritis, as assessed by measurement of protein in the urine (i.e., proteinuria).

At 8 months of age, most NZBxNZW F1 mice, an autoimmune disease mouse model, will have developed high titers of autoantibodies and show evidence of nephritis. Serum antibodies and proteinuria in these NZBxNZW F1 mice was assessed, along with their controls, longitudinally over the following 4 months. Organs, such as kidneys and spleens, were dissected from the animal at the end of the experimental period to assess whether the experimental approach inhibited or reversed progression of autoimmune disease.

An additional consideration of the present methods is that CTL infusion into diseased NZBxNZW F1 mice may elicit side effects. For example, severely ill cancer patients treated with anti-CD19 CTLs showed varying, usually transient, side effects that ranged in severity from fatigue, nausea, and diarrhea to liver enzyme perturbation and acute renal failure. A particular concern regarding side effects of the present method of treatment of autoimmune disease is the impairment of kidney function. Increased B cell death may enhance nuclear antigen deposition in the glomeruli of kidneys, and therefore, particular attention to measure proteinuria in treated mice during the first 10 days following initiation of the anti-CD19 therapy in diseased animals.

### Example 3: Ex Vivo Anti-CD19 Expression in Mice and Human Cells

To evaluate alternative gene delivery vehicles, we transduced the anti-CD19 CAR into splenic CD8+ T cells via retroviruses engineered to express either the amphotropic surface glycoprotein or the pantropic VSV-G glycoprotein or via VSV-G-pseudotyped lentivirus (see FIG. 3A). The infections achieved over 90% expression (approximately 93.03%) in cells from the human Jurkat T cancer cell line (see FIG. 5A). In addition, the infections showed between 5-20% expression of anti-CD19 CAR in primary T cells from NZBxNZW F1 mice (see FIG. 5B). Following a 5-6 day of ex vivo cell expansion in culture, the anti-CD19 CAR T cells were infused into the NZBxNZW F1 animals. Negative control mice were also infused with non-transduced T cells, such as non-transduced syngeneic CD8⁺ T cells.

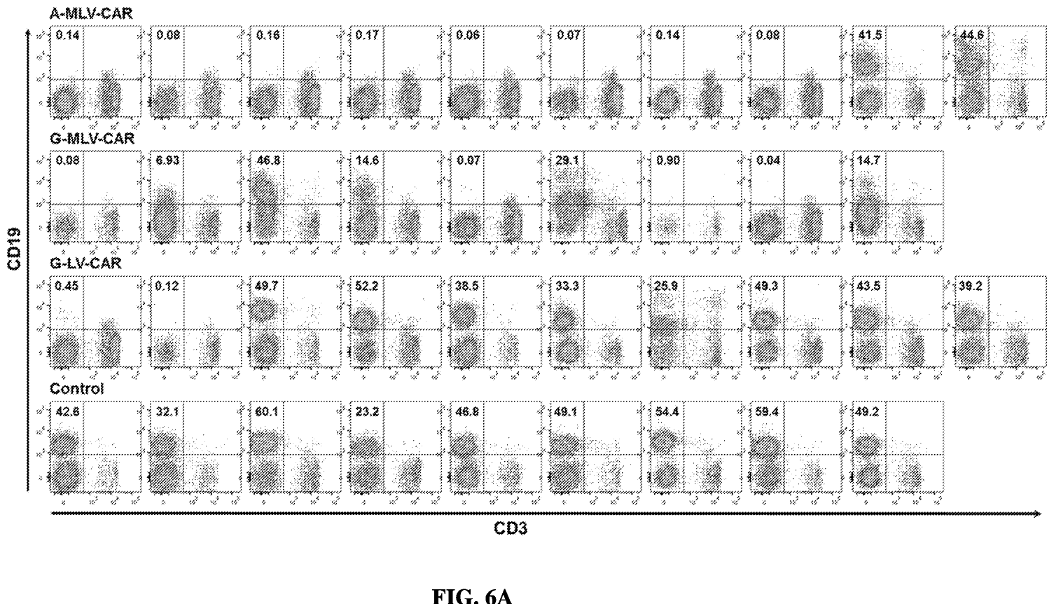

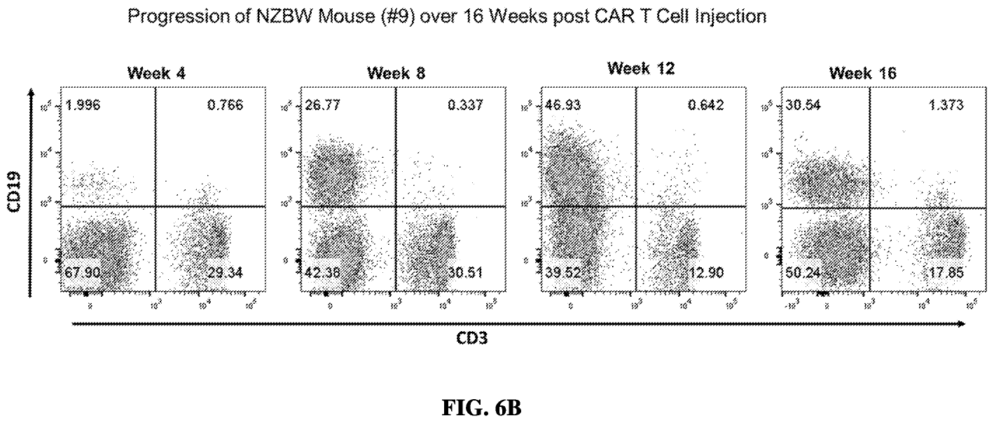

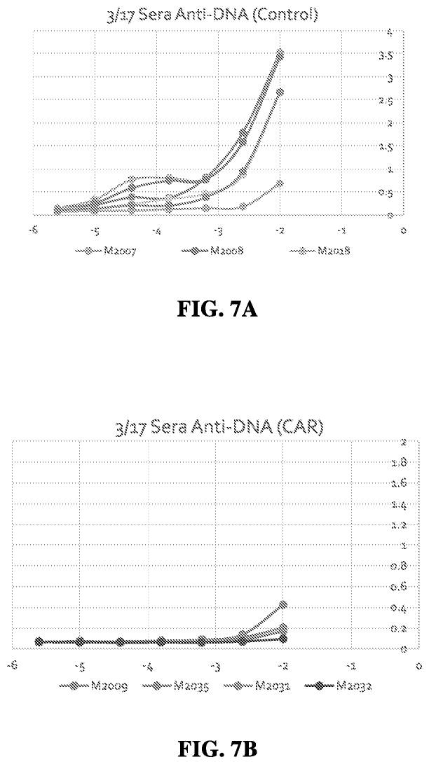

11 weeks post-treatment of the approximately 40 NZBxNZW mice, whole blood was extracted from the mice segregated into 4 treatment groups comprising about 9-10 mice each (see FIG. 7A). Establishing a frequency or quantity of CD19⁺ lymphocytes at or below 0.2% of total lymphocytes as being a minimum cutoff at, 8 of 10 mice receiving the anti-CD19 CAR T cells via the amphotropic retrovirus vector showed depleted CD19⁺ B cells having only about 5-20% or about 6-17% of cells identified as B cell or CD19+ by flow cytometry (see FIG. 6A). In contrast, CD19⁺ B cell depletion occurred in only 4 of 9 11 weeks post-treatment of the approximately 40 NZBxNZW F1 mice receiving T cells transduced with VSV-G-pseudotyped retrovirus (see FIG. 6A). In addition, only 2 of 10 mice receiving lentivirus-transduced T cells showed significant B cell depletion in mice (see FIG. 6A). This data outcome confirmed that some mice experimental groups were defined by the success or failure of CD19⁺ B cell depletion.

CD19⁺ B cell depletion was stably and consistently sustained in treated NZBxNZW mice for over 1 year. In fact, in all but one mouse, the B cell depletion was stably sustained over for a time period including but not limited to at least 12 months, 18 months, 24 months, 48 months, indefinitely, and for the remainder of the lifetime of the mouse. However, one NZBxNZW F1CAR-T treated mouse recovered significant CD19⁺ B cell expression as soon as 16 weeks after treatment (see FIG. 6B).

At 4 weeks post-treatment, the single NZBxNZW F1 mouse demonstrating the unstable B cell depletion phenotype, showed only about 2% of CD19+ B cells (see FIG. 6B). However, by 8 weeks post-treatment, the mouse demonstrated about 27% CD19+ B cells, which was up to as high as about 44% at week 12 and seemingly decreasing back down to about 31% by week 16 after treatment. The unique and unstable B cell depletion phenotype demonstrated by this mouse was unexplained and unrepeated in any other experimental subjects.

### Example 4: Improvements in Clinical Manifestations of Lupus—Anti-DNA Titers

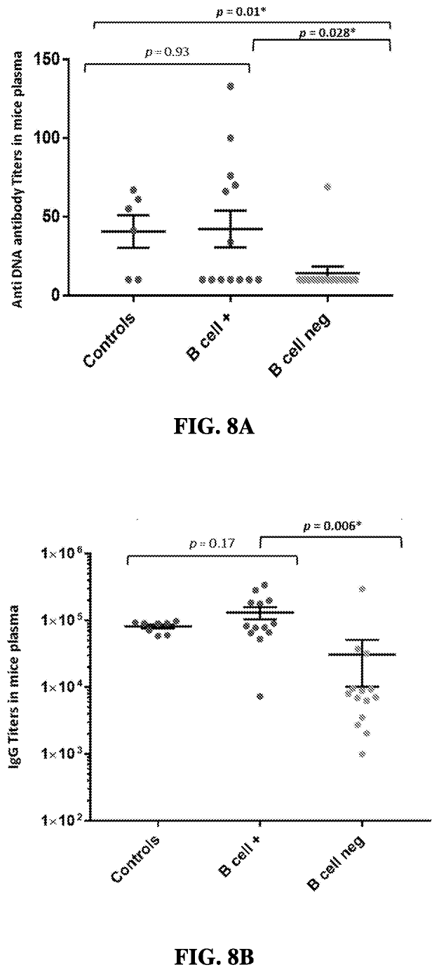

To confirm whether the absence of B cells in CD19 CAR T cell-treated NZBxNZW F1 mice led to an improvement in clinical manifestations of lupus, such as autoantibody titers, normalized kidney function, and/or extended the life span relative to control NZBxNZW F1 mice or CD19-sufficient recipients, CD19-depleted lupus mice from the three viral vector treatment groups were combined for subsequent analyses. Sera from the treated and control NZBxNZW F1 mice revealed a dramatic decline in anti-dsDNA IgG titers and an even larger decrease in total IgG concentrations in CD19-depleted mice relative to controls. FIGS. 7A and 8B show measurement of anti-DNA IgG titers in control NZBxNZW F1 mice (see FIG. 7A) and in CAR-CTL-treated littermates (see FIG. 7B). Three months after administration of anti-CD19 CAR CTL, the IgG anti-DNA titers remained near background levels.

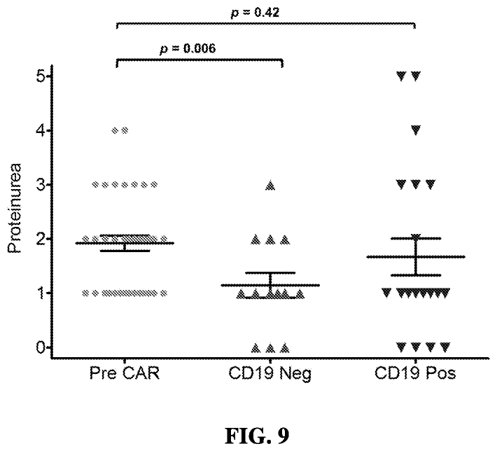

Additionally, total plasma IgM and IgG and anti-DNA autoantibody from CD19 B cell depleted (CD19d) and CD19 B cell sufficient (CD19s or CD19+) mice were analyzed and compared using an enzyme-linked immunosorbent assay to control mice (ELISA; see FIGS. 8A and 8B). Total IgG and anti-DNA IgG decreased precipitously to near background levels in both mice strains and remained at the reduced or decreased levels. These results were in complete correlation with the sustained B cell depletion observed in CAR-T infused NZBxNZW F1 mice (see FIGS. 8B and 9).

In contrast, non-transduced T cell infused control mice had progressive increases in plasma IgG and anti-DNA IgG autoantibody consistent with normal autoimmunity in NZBxNZW F1 mice (see FIGS. 8A and 8B). Importantly, plasma IgM and anti-DNA IgM showed more moderate decreases over time and remained clearly above background (see FIGS. 8A and 8B).

### Example 5: Improvements in Clinical Manifestations of Lupus—Kidney Proteinuria

To determine whether B cell depletion and the correlating reduction in anti-DNA autoantibody titers observed in CAR T-treated NZBxNZW F1 mice also yielded improvements in lupus nephritis, proteinuria was measured. Proteinuria is the measure of the amount of protein in the urine of a subject. Increased and/or high amounts of protein deposits in the urine of a subject indicates a problem of failure of the filtering system of the kidneys. Accordingly, when the kidneys of a subject are not functioning properly, for example as a side effect or symptom of a larger condition, such as an autoimmune disease (e.g., lupus), proteinuria serves as an indicator of the presence and/or progression of the disease.

Notably, proteinuria, was detectable in 7-month-old NZBxNZW F1 mice at the start of treatment. Thus, animals were symptomatic prior to treatment. Nonetheless, proteinuria improved in symptomatic NZBxNZW F1 mice after treatment and CD19⁺ B cell depletion occurred (see FIG. 9). In contrast, proteinuria remained high in CD19⁺ B cell-sufficient NZBxNZW F1 mice (see FIG. 9).

### Example 6: Improvements in Clinical Manifestations of Lupus—Kidney Glomeruli and Spleen

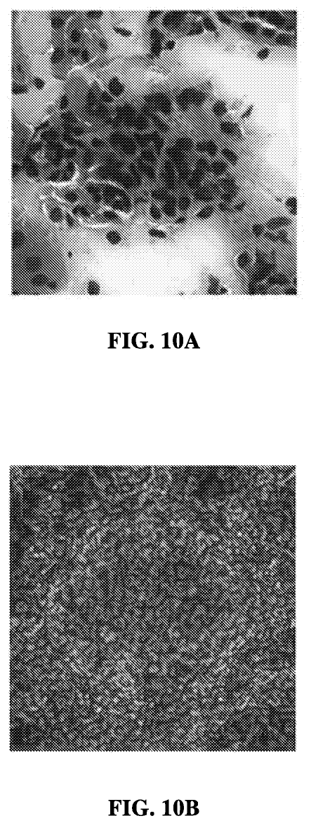

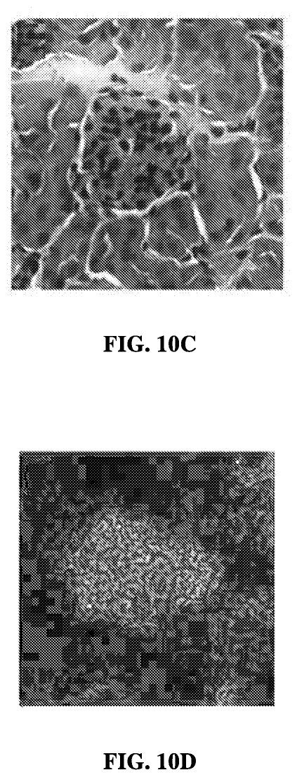

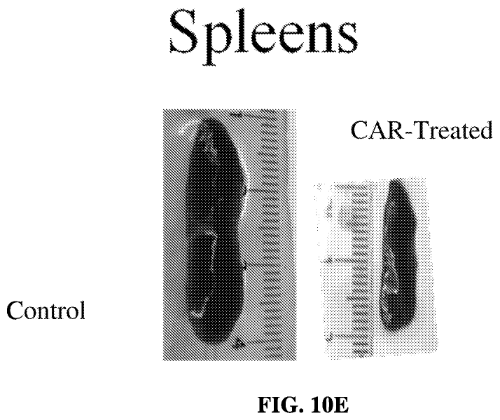

Histopathological evaluation of NZBxNZW F1 mice kidney cryosections were evaluated by H & E staining at 6 months post-treatment (see FIGS. 10A-10D). Untreated NZBxNZW F1 mice tissues were compared to CAR T-treated mice tissues to confirm whether CAR-T treatment reduced the size and cellular infiltrate in kidney glomeruli (see FIGS. 10A and 10C, respectively). In addition, the untreated and treated kidney tissue sections were observed to compare the presence of IgG deposits (see FIGS. 10B and 10D, respectively).

The results indicate that untreated NZBxNZW F1 mice showed enlarge, hypercellular glomeruli (see FIG. 10A) due to the improper functioning of the mice kidneys causing protein deposits and filtrate to collect in the glomeruli, enlarge, and/or expand. In contrast, the CAR T-treated mice glomeruli of FIG. 10C are have a normal appearance.

In addition, inflammation is a major symptom of autoimmune diseases, which causes an influx of cells, including immunoglobulin (IgG) complexes, into the kidneys. This influx of cells leads to damage to the basement membrane of glomeruli. FIG. 10B shows significant immunoglobulin (IgG) deposits in the kidneys of untreated mice, wherein the CAR-T treated mice largely prevented IgG kidney deposits, which showed fewer IgG deposits, in both autoimmune strains of treated mice, NZBxNZW F1 (see FIG. 10D) and MRL/lpr (data not shown).

Finally, FIG. 10E demonstrates that anti-CD19 CAR-treated NZBxNZW F1 mice had a normal sized spleen of about 2 cm in length. In contrast, the spleen of untreated control mice were significantly enlarged to about 3 cm in length (see FIG. 10E). CAR-T-treated mice demonstrated reduced splenomegaly with about a 30% reduction in size compared to non-treated control mice (see FIG. 10E). Accordingly, these data demonstrate that the CAR T-treated mice are able to sufficiently prevent, delay, suppress, and/or reverse clinical manifestations of autoimmune diseases, such as lupus.

### Example 7: Improvements in Clinical Manifestations of Lupus—Life Expectancy

The methods of the present disclosure have been demonstrated to effectively and efficaciously mitigate several symptoms and/or clinical manifestations of lupus (e.g., decreased or depletion of B cells, anti-DNA titers, reduced kidney proteinuria, and reduced kidney glomeruli and IgG deposits). Importantly, the claimed methods also improve the life expectancy of a subject treated as described herein.

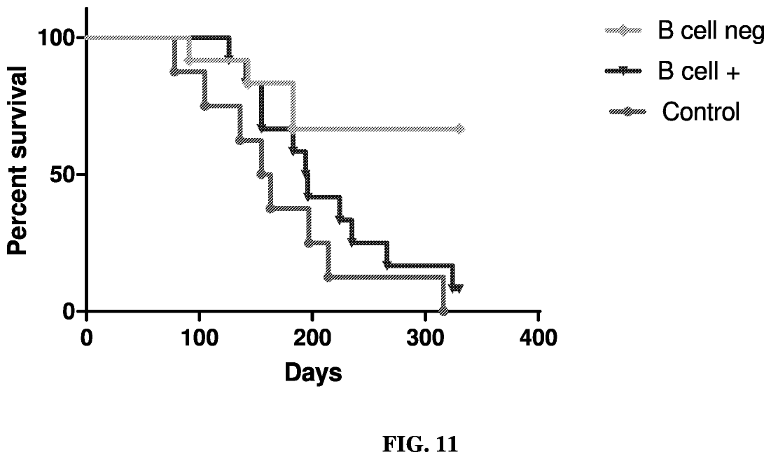

FIG. 11 demonstrates that CAR-Treated NZBxNZW F1 mice have stable B cell depletion significantly outlived control mice over a study time period of about 12 months (i.e., 320 days or more). Moreover, about 60-65% of CAR T-treated NZBxNZW F1 mice lived past the time when all control NZBxNZW F1 mice had died. Thus, these data demonstrate that treatment of a subject with the claimed methods significantly improves the life expectance and mortality rate of the treated subject as compared to control subjects that were not treated with the engineered cytotoxic T cells of the present methods.

Notably, both NZBxNZW F1 mice and MRL/lpr mice had increased lifespans after anti-CD19 CAR-T infusions. Most NZBxNZW F1 mice treated with anti-CD19 CAR-T cells lived until about 18 months of age. Generally, MRL/lpr mice treated with anti-CD19 CAR-T cells lived for over one year (see FIG. 11). However, both NZBxNZW F1 mice and MRL/lpr mice treated with anti-CD19 CAR-T cells outlived untreated or control-treated NZBxNZW F1 mice and MRL/lpr mice, respectively.

### Example 8: Persistence and Stability of B Cell Depletion in Treated Mice Over Time

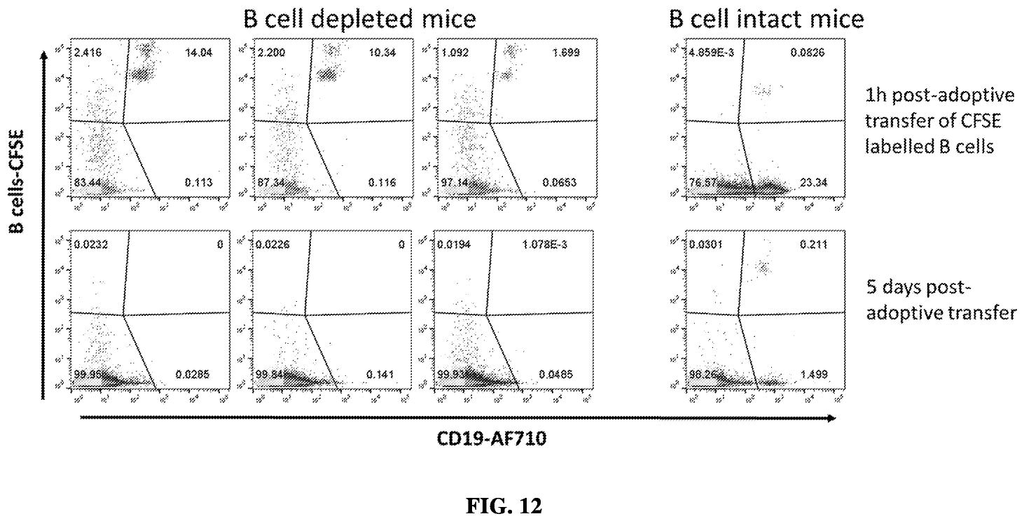

There are a multitude of reasons why B cell depletion or loss of B cell function and/or activity may occur subsequent to CAR T-cell treatment. Accordingly, anti-CD19 CAR T cell function activity was confirmed to be present after 5 months, 6 months, and 7 months of testing the cells in vivo by adoptive transfer of CFSE-labeled B cells (see FIG. 12). One hour was not sufficient enough time to observe depletion of B cells in control or post-CAR-T treated mice (see FIG. 12). However, in CAR-T-treated mice, transferred/labeled B cells were undetectable 5 days after transfer, whereas CFSE-labeled B cells were clearly present in control mice 5 days after treatment. B cell function in the CAR-treated mice was functional and active well beyond 7 months after treatment.

### Example 9: Confirmation of Stable B Cell Depletion in MRL/lpr Mice

The present methods described herein were further tested an additional mouse strain. Similar to the NZBxNZW F1 mice, MRL/lpr mice are additional mouse models for autoimmune diseases, such as lupus. However, the MRL/lpr mice strain is a much more severe lupus model. More specifically, female MRL/lpr mice develop clinical manifestation of lupus disease faster and more severely than other mouse strains, such as the NZBxNZW F1 mice.

MRL/lpr mice were treated with the cytotoxic T cell treatment method described herein to investigate depletion of CD19⁺ B cells. Twenty-three, 2-month-old MRL/lpr female mice were used in the study, 12 were treated and 11 were treated with a control (FIG. 2B). As controls, mice were infused with non-transduced syngeneic CD8⁺ T cells. Cells from untreated and CAR-treated cells from MRL/lpr mice were analyzed via flow cytometry for the presence and/or depletion of B cells.

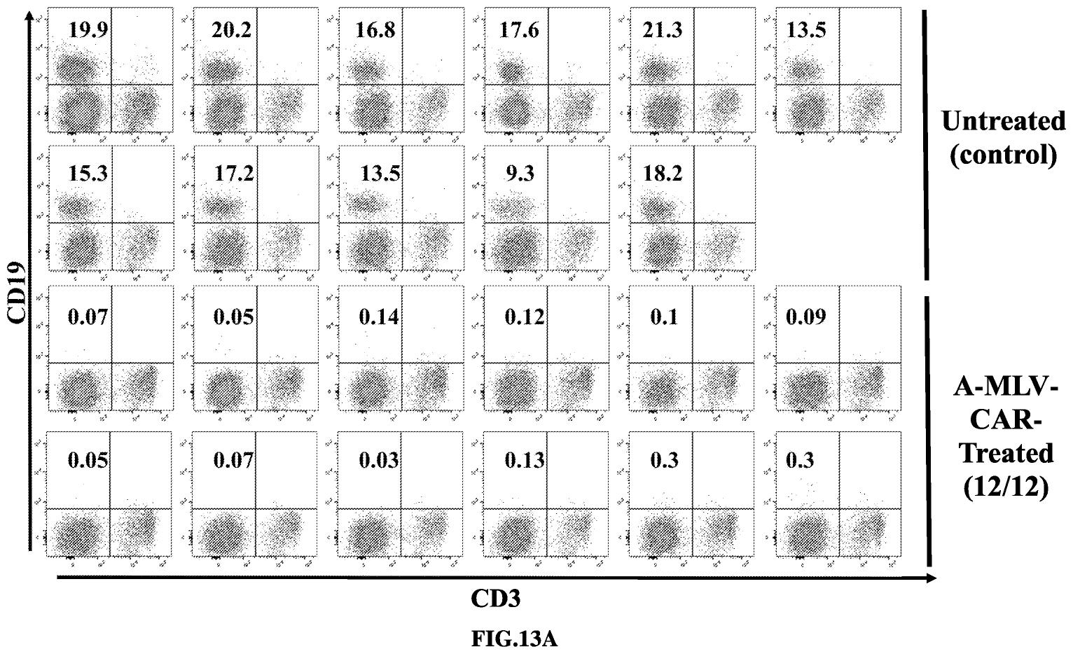

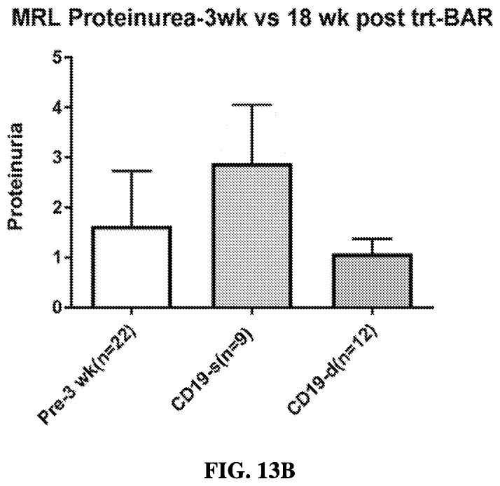

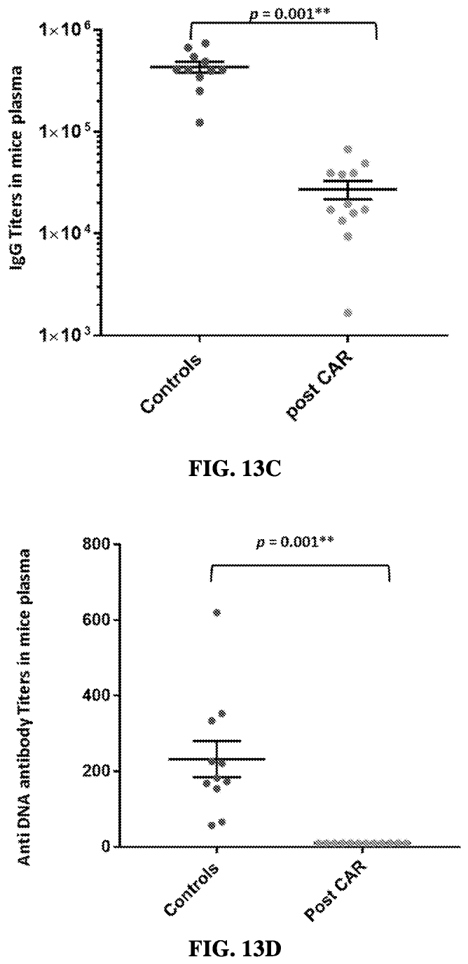

FIG. 13A demonstrates that 100% of the dozen CAR-treated MRL/lpr mice showed a significant depletion of B cells. More specifically, the amount of CD19+ B cells observed after CAR T treatment of MRL/lpr mice ranged from about 0.01-0.5%, and more specifically from about 0.05-0.14% of CD19+ B cells. In contrast 100% of the 11 untreated control MRL/lpr mice showed a significant presence of CD19+ B cells. However, the amount of CD19+ B cells observed in the untreated control MRL/lpr mice ranged from about 9.3-19.9% of CD19+ B cells, which was significantly higher than the CAR T-treated MRL/lpr mice. Thus, a steady increase in the population of B cells in the untreated control mice was observed (9.3-19.9% of CD19+ B cells) as compared to the negligible number of B cells that were observed for CAR T-treated MRL/lpr mice (0.05-0.14% of CD19+ B cells).

Proteinuria was also observed to be reduced in CAR T-treated MRL/lpr mice, but increased over time in non-treated mice (see FIG. 13B). At 18 weeks post-CAR treatment, CD19-deficient (CD19d) mice showed a significant increase in proteinuria as compared to the untreated control mice and CD19 sufficient (CD19s) mice, which had the lowest amount of proteinuria observed.

In contrast, non-transduced T cell infused control mice had progressive increases in plasma IgG and anti-DNA IgG autoantibody consistent with normal autoimmunity in MRL/lpr mice (see FIGS. 13C and 13D). Importantly, plasma IgG and anti-DNA IgM showed no detectable biding of CAR-T cells to DNA and reduced levels of IgG (see FIG. 13C). In addition, the anti-DNA marker, which is very important for lupus detection in mice and humans, showed complete B cell depletion of CAR-treated cells (see FIG. 13D).

Unexpectedly, by 18-19 weeks after anti-CD19 CAR-T cell infusion, MRL/lpr mice and NZBxNZW F1 mice had mean total plasma IgM levels that were similar to those in non-transduced controls. Consequently, circulating B lymphocytes were investigated by flow cytometry with anti-IgM antibody (see FIGS. 13C and 13D). Control mice of both strains had an lgM^{hi} and an IgM^{lo} subpopulation of IgM positive B cells (see FIG. 13C). The IgM^{lo}, but not the lgM^{hi}, subpopulation of B cells had recovered in CAR-T-treated MRL/lpr mice by 5 weeks of age. These cells remained CD19-negative. Thus, the persisting IgM^{lo} population may be the source of IgM antibodies in circulation.

### Example 10: Improvements in Clinical Manifestations of Lupus in MRL/lpr Mice—Alopecia

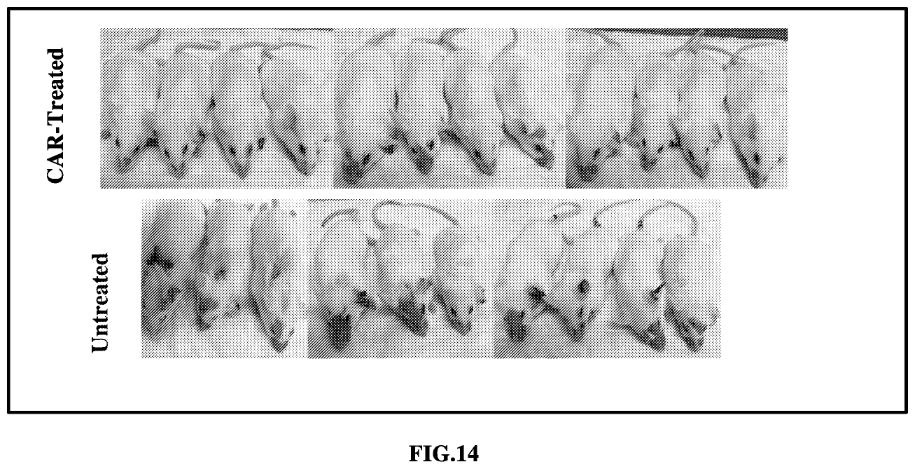

In MRL/lpr mice, autoimmune diseases, such as lupus, also manifest in a skin disorder that begins with alopecia and progresses to skin lesions, scabs, and scarring. To examine the skin pathology in CAR-T treated and control mice, we compared skin tissue sections by microscopy. At eight months, when the control MRL/lpr mice had neutrophilic infiltrates and inflammation progressing to fibrosis, the CD19⁺ B cell-depleted MRL/lpr mice had nearly normal skin architecture and minimal alopecia (see FIG. 14).

Depletion of CD19⁺ B cells by anti-CD19 CAR CD8⁺ T cells effectively suppressed autoantibody production in two mouse models and prevented, deferred, suppressed, or reversed clinical manifestations of autoimmune diseases, such as lupus disease. The results of the present methods contradict the results of the anti-CD20 antibody treatments (i.e., Rituximab) in the same mouse models, which showed diminishing efficacy over time. The increased efficiency of anti-CD19 CAR T cells lies in the specificity and type of treatment used.

CD19 is a B cell surface marker that is intimately involved in B cell signaling and contributes to B1 B cell development in the marginal zone of the spleen. Moreover, CD19 is expressed in more differentiated B cells than CD20, including on some plasma cells. As such, anti-CD19 CAR T cells may target B cells that are directly responsible for autoantibody production.

Remarkably, the depletion of CD19-positive B cells did not eliminate all IgM-positive B cells, and CAR-treated MRL/lpr mice exhibited persistent plasma IgM. It is not clear whether residual IgM-positive cells are equivalent to the B1 B cells that are abundant in CD19 knock-out mice, but an interesting parallel exists with anti-CD19 CAR-treated patients who often demonstrate a return of antibody in serum despite continued CD19-B cell depletion.

Anti-CD19 CAR T cells achieve outcomes that are superior to prior art antibody treatments because CAR T cells proliferate in the host subject, and thus offer permanent suppression of CD19 B cell production, which is an unexpected result of the present methods. In addition, cell-based therapies ensure that CD19 B cells are depleted from different tissues of the subject to which CAR T cells have access, such as the blood, spleen, and bone marrow of treated mice (data not shown). Thus, B cell depletion starts as soon as CD19-positive, pro-B cells arise in the bone marrow and extends to more mature CD19⁺ B cells present in the spleen or the blood.

Notably, B cells, the targets of the presently disclosed CAR therapy, are different in cancer versus autoimmune disease. In cancer, B cells are malignant, and therefore pose a threat to the host because of their hyper-proliferation. In autoimmune disease, B cells escape tolerance and produce autoantibodies that attack the host. It is the product of the B cells (i.e., the antibody or autoantibody), which is the greatest danger to the health of the autoimmune subject or patient. Therefore, determining whether the presently describe method of treatment of autoimmune diseases, comprising anti-CD19 CAR T cells, would actually be effective against autoreactive B cells causing autoimmune diseases, such as lupus, was not clear or obvious because of the fundamental difference in the type of pathology that B cells induce in autoimmune disease versus in cancer.

Further, in autoimmune disease, autoreactive B cells may increase, significantly, in numbers due to the disrupted regulation by immune tolerance. However, the main and proximal cause of many and/or most autoimmune diseases (e.g., lupus) is the production of autoantibodies by the B cells. Autoantibodies recognize specific antigen substances in the body of a subject in such a manner that their binding damages tissues or impairs their function. B cell depletion, or the reduction and/or depletion in the number of B cells present in a subject or a patient, is a direct quantitative measure of successful autoimmune disease therapy. However, it should be noted that even a small number of autoreactive B cells can continue to secrete autoantibodies that perpetuate autoimmune disease and/or inflict clinical manifestations of autoimmune disease on a patient, such as tissue damage. Therefore, an important measure of a truly successful therapy for autoimmune disease (i.e., efficacy of the immunotherapy) is the effective, complete, stable, and lasting depletion of autoantibody producing B cells from different sites in the body of a subject, which stops further tissue damage and/or disease progression.

Importantly, it should also be noted that T cells, the “executioners” of the cytotoxic (killing) process used in the present methods, are also affected by the autoimmune disease process, as they also become autoreactive, similar to B cells. Accordingly, use of these cytotoxic T cells in the treatment of autoimmune diseases, as described herein, was not obvious since it was not clear or known whether autoreactive T cells would sufficiently and/or efficaciously carry out depletion of autoreactive B cell, which are causative of autoimmune diseases, in order to reduce, reverse, and/or prevent clinical manifestations of autoimmune disease in a subject or patient.

Only the inventors of the subject application performed the necessary experiments to demonstrate that the claimed methods could be performed safely and could be stably maintained over an extended period of time, including indefinitely. One precaution incorporated by the inventor was to enrich B cells with CD8⁺ T cells before transduction with a virus encoding the anti-CD19 CAR.

CD8⁺ T cells are naturally cytotoxic, and are therefore more likely to be well-suited, following transduction, for B cell depletion. In cancer therapies, CAR-T cells often are generated from a mixture of both CD4⁺ and CD8⁺ T cells. In autoimmune disease, CD4⁺ T cells, which are helper T cells, can aggravate autoimmune disease by helping, i.e. stimulating autoreactive B cells. Therefore, in the claimed method embodiments, CD4+ T cells may not be used. In a further method embodiment, CD4+ T cells are not used. Instead, B cell populations are enriched with CD8+ T cells, which are the exemplary cytotoxic T cell to engineer with a CAR in order to successfully perpetuate B cell depletion as specified by the presently claimed methods.

Additionally, the present methods comprising reinfusion or reinjection of engineered CAR-T cells into a subject are superior to prior art antibody injections because cytotoxic T cells induce target cell death by a direct mechanism. In contrast, antibody-mediated cytotoxicity requires the build-up of bound antibody for opsonization and subsequent target cell phagocytosis or complement-dependent lysis. In an autoimmune disease, such as lupus, the increased abundance of endogenous antibodies leads to competition with the therapeutic protein that further decreases its effectiveness. Thus, prior art anti-CD20 antibody treatment can only be effective if administered repeatedly and at high doses to autoimmune mice. This is likely the reason for the relatively poor response to Rituximab in patients with lupus, and establishes a distinct benefit of the claimed methods, which only require a single administrated of the engineered CAR T cells.

The long-term efficacy of B cell depletion in our study may have benefited from the use of a second generation CAR in which the CD3ζ (zeta) signaling domain was attenuated. Previous studies demonstrated that dampened CAR signaling led to longer in vivo persistence of CAR T cells. In addition, the present inventors determined that amphotropic MLV retrovirus is a preferable means of CAR gene delivery to mouse CD8+ T cells, as compared to VSV-G pseudotyped RV or LV.

The second generation CAR construct combined with the amphotropic MLV retrovirus resulted in stable CAR expression that continued for over one year in NZBWF1 mice. Functionality of the CAR T cells remained stable because the CAR treated mice rapidly depleted transferred autologous CD19 B cells, whereas such cells persisted in control animals (FIG. 12). Moreover, splenic CD8+ T cells from CAR-treated MRL/lpr mice were also transferred to a second cohort of recipient mice and successfully effectuated B cell depletion in the new hosts (data not shown).

In humans, this combination of markers serves to identify a stem cell-like T cell memory population, which has the capacity for self-renewal and differentiation into effector T cells. It will be important to demonstrate that the CD62L/CD44 double-positive cells identified here in mice also share this combination of features identified herein because these T cells are particularly potent in suppressing their therapeutic target cells.

In summary, advances in CAR T cell technology promise new treatments that will become more effective and easier to administer to subjects, such as mice and humans. This makes applications of CAR T cells in human patients with lupus a likely future therapy. Despite differences between the mouse and human immune system and the fact that mice do not fully model all characteristics of lupus, our data support initial testing of anti-CD19 CAR T cells in human lupus patients.

Various modification and variation of the described methods and compositions of the present application will be apparent to those skilled in the art without departing from the scope and spirit of the present application. Although the present application has been described in connection with specific preferred embodiments, it should be understood that the present application as claimed should not be unduly limited to such specific embodiments. Indeed, various modifications of the described modes for carrying out the present application that are obvious to those skilled in the relevant fields are intended to be within the scope of the following claims.

1. A method of treating an autoimmune disease in a subject, the method comprising:
administering a plurality of engineered cytotoxic T cells to the subject,
wherein each of the plurality of engineered cytotoxic T cells comprise a recombinant vector that expresses a chimeric antigen receptor,
depleting the number of antibody-producing cells in the subject,
improving one or more clinical manifestations of the autoimmune disease in the subject, and
treating the autoimmune disease in the subject.

2. The method of claim 1, wherein the autoimmune disease is Systemic Lupus Erythematosus (SLE).

3. The method of claim 1, wherein the subject is a mammal.

4. The method of claim 3, wherein the mammal is a mouse.

5. The method of claim 3, wherein the mammal is a human.

6. The method of claim 1, wherein the one or more clinical manifestations of the autoimmune disease in the subject comprises an increase in B cells.

7. The method of claim 1, wherein the antibody-producing cells are B cells.

8. The method of claim 1, wherein the chimeric antigen receptor (CAR) comprises anti-CD19.

9. A method of treating one or more clinical manifestations of lupus in a subject, the method comprising:
administering a plurality of engineered cytotoxic T cells to the subject, wherein each of the plurality of engineered cytotoxic T cells comprise a recombinant vector that expresses a chimeric antigen receptor,
reducing or depleting the number of antibody-producing cells in the subject,
preventing, delaying, or reversing one or more clinical manifestations of lupus in the subject, and
treating the one or more clinical manifestations of lupus in the subject.

10. The method of claim 9, wherein the lupus is Systemic Lupus Erythematosus (SLE).

11. The method of claim 9, wherein the subject is a mammal.

12. The method of claim 11, wherein the mammal is a mouse or a human.

13. The method of claim 9, wherein the one or more clinical manifestations of lupus in the subject comprises an increase in B cells.

14. The method of claim 9, wherein the chimeric antigen receptor (CAR) comprises anti-CD19.

15. A method of monitoring efficacy of lupus treatment in a subject, the method comprising:
measuring one or more clinical manifestations of lupus in the cells and/or tissues of a subject prior to treatment administration,
administering a treatment construct comprising a plurality of engineered cytotoxic T cells to the subject, wherein each of the plurality of engineered cytotoxic T cells comprise a recombinant vector that expresses a chimeric antigen receptor, at least one hour after treatment administration,
remeasuring the one or more clinical manifestations of lupus in the subject,
assessing the one or more clinical manifestations of lupus by determining the difference between the cells and/or tissues of the subject prior to treatment administration compared to the cells and/or tissues of the subject after treatment administration.

16. The method of claim 15, wherein the lupus is Systemic Lupus Erythematosus (SLE).

17. The method of claim 15, wherein the subject is a mammal.

18. The method of claim 17, wherein the mammal is a mouse or a human.

19. The method of claim 15, wherein the one or more clinical manifestations of lupus in the subject comprises an increase in B cells.

20. The method of claim 15, wherein the chimeric antigen receptor (CAR) comprises anti-CD19.
